(19) United States
(12) Patent Application Publication
(10) Pub. No.: US 2026/0232933 A1
(43) Pub. Date: Aug. 13, 2026

(54) DOSE COUNTER SUBASSEMBLIES FOR USE IN DRY POWDER INHALERS

(71) Applicant: TRANSPIRE BIO INC., Weston, FL (US)

(72) Inventors: Thomas Alexander EARWAKER, Leamington Spa (GB); Zoe Georgina ARMSTRONG, Leamington Spa (GB); Michael Ray NICHOLSON-LEWIS, Leamington Spa (GB); Matthew Meredith JONES, Warwick (GB); Jianye WANG, St. Albans (GB); James Alexander SENIOR, Warwick (GB)

(21) Appl. No.: 19/153,794

(22) PCT Filed: Feb. 1, 2024
(86) PCT No.: PCT/US2024/014017

§ 371 Date: Aug. 5, 2025

Related U.S. Application Data
(60) Provisional application No. 63/483,393, filed on Feb. 6, 2023.

(51) Int. Cl. A61M 15/00

(52) U.S. Cl. CPC A61M 15/0073; A61M 15/0026; A61M 15/0051; A61M 2202/064

(57) ABSTRACT

Inhaler devices for delivering dry powdered medicament from at least one blister strip. The inhaler device includes an actuator for operating a dispensing mechanism of the inhaler device. Dose counter subassemblies for use in the inhaler devices include a first count wheel and a second count component. The first count wheel and the second count component collectively display a number of doses remaining within the inhaler device or a number of doses delivered by the inhaler device. The first count wheel is configured to be rotated by the dispensing mechanism, and the second count component is driven by the first count wheel. The second count component intermittently rotates or translates once per revolution of the first count wheel.

### CROSS REFERENCE OF RELATED APPLICATIONS

This application claims the benefit of U.S. Provisional Patent Application No. 63/483393, filed Feb. 6, 2023, which is hereby incorporated by reference in its entirety for all purposes.

### FIELD OF THE INVENTION

The invention relates in general to an inhaler device, and more particularly to dose counter mechanisms or subassemblies for use in an inhaler device configured to dispense dry powder medicament from one or more blister strips.

### BACKGROUND OF THE INVENTION

Medicaments may be administered to a patient by inhalation using dry powder inhaler devices. Such devices are often used for the treatment and prophylaxis of respiratory diseases, including but not limited to asthma and chronic obstructive pulmonary disease (COPD). A dry powder inhaler device may include a blister strip containing several discrete doses of powdered medicament. Such devices typically contain a mechanism such as piercing means for accessing a medicament dose by opening one or more blister pockets. The powdered medicament can then be accessed by the device and inhaled via the device by the user.

Devices of this kind are often used regularly by patients and can form an important part of their treatment. It is therefore valuable for the patient to know how many doses are remaining in the device, so that they are aware when a device is empty and can plan for replacement devices. Further, it is valuable to ensure that the devices are easy to operate to increase the chance of correct use of the device and effective treatment. Minimizing the number of operating steps and giving clear visual/tactile feedback is therefore advantageous. The present invention relates to mechanisms for advancing the discrete medicament doses of an inhaler device and also relates to counter mechanisms for displaying the number of doses remaining in an inhaler device.

### BRIEF SUMMARY OF THE INVENTION

According to a first embodiment hereof, the present disclosure provides a dry powder inhaler device including an actuator for operating a dispensing mechanism of the dry powder inhaler device, a first count wheel configured to be rotated by the dispensing mechanism, and a second count component disposed adjacent to the outer circumferential surface or side of the first count wheel. The first count wheel includes a single tooth extending radially outward from an outer circumferential surface or side thereof. The second count component includes a plurality of notches on an outer surface or side thereof, each notch extending radially inward and each notch configured to mate with the single tooth. The single tooth of the first count wheel engages one of the plurality of notches of the second count component once per revolution of the first count wheel to intermittently rotate the second count component, the first count wheel and the second count component being configured to rotate in opposing directions. The first count wheel and the second count component collectively display a number of doses remaining within the inhaler device or a number of doses delivered by the inhaler device.

In an aspect of the first embodiment, and in combination with any other aspects herein, the disclosure provides that the actuator is a mouthpiece cover.

In an aspect of the first embodiment, and in combination with any other aspects herein, the disclosure provides that the first count wheel rotates about a first axis and the second count component rotates about a second axis, the second axis being parallel to and spaced apart from the first axis.

In an aspect of the first embodiment, and in combination with any other aspects herein, the disclosure provides that the plurality of notches includes four notches.

In an aspect of the first embodiment, and in combination with any other aspects herein, the disclosure provides that a segment of the outer surface or side of the second count component extending between two adjacent notches of the plurality of notches is concave and forms an arc-shaped indent that is coincident with the outer circumferential surface or side of the first count wheel.

In an aspect of the first embodiment, and in combination with any other aspects herein, the disclosure provides that the outer surface or side of the second count component includes a plurality of segments, each segment extending between two adjacent notches of the plurality of notches. Each segment is concave and forms an arc-shaped indent that is coincident with the outer circumferential surface or side of the first count wheel.

In an aspect of the first embodiment, and in combination with any other aspects herein, the disclosure provides that the first count wheel is an annular component.

In an aspect of the first embodiment, and in combination with any other aspects herein, the disclosure provides that the second count component is a non-annular component.

In an aspect of the first embodiment, and in combination with any other aspects herein, the disclosure provides that an indicia-displaying surface of the first count wheel includes ‘ones’ digits disposed thereon and an indicia displaying surface of the second count component includes ‘tens’ digits disposed thereon.

In an aspect of the first embodiment, and in combination with any other aspects herein, the disclosure provides that the indicia-displaying surfaces of the first count wheel and the second count component are coplanar.

In an aspect of the first embodiment, and in combination with any other aspects herein, the disclosure provides that the indicia-displaying surfaces of the first count wheel and the second count component are not coplanar, with the indicia displaying surface of the second count component disposed closer to a display window of the inhaler device than the indicia displaying surface of the first count wheel.

In an aspect of the first embodiment, and in combination with any other aspects herein, the disclosure provides that the second count component is configured to rotate in a first direction and the first count wheel is configured to rotate in a second opposing direction.

In an aspect of the first embodiment, and in combination with any other aspects herein, the disclosure provides that the second count component includes a single flag in place of a zero, the single flag being a colored block with no digits thereon.

In an aspect of the first embodiment, and in combination with any other aspects herein, the disclosure provides that the second count component includes a double flag, the double flag being a colored block with no digits thereon. The double flag is configured to cover a digit of the first count wheel when no doses remain within the inhaler device.

In an aspect of the first embodiment, and in combination with any other aspects herein, the disclosure provides that the outer circumferential surface or side of the first count wheel is generally circular with a clearance gap around the single tooth.

In an aspect of the first embodiment, and in combination with any other aspects herein, the disclosure provides that the second count component is stationary when the single tooth of the first count wheel is not engaged within one of the plurality of notches of the second count component.

In an aspect of the first embodiment, and in combination with any other aspects herein, the disclosure provides that the first count wheel includes detent wheel having a plurality of notches. The dry powder inhaler device further includes a housing and a flexible arm extending from a housing, the flexible arm including a detent thereon, the detent being configured to be received within a notch of the plurality of notches of the first count wheel.

In an aspect of the first embodiment, and in combination with any other aspects herein, the disclosure provides that the flexible arm is configured to drive and secure the first count wheel within a target position.

In an aspect of the first embodiment, and in combination with any other aspects herein, the disclosure provides that the dry powder inhaler device further includes a housing and a flexible arm extending from a housing, the flexible arm including a detent thereon, the detent being configured to be received within a notch of the plurality of notches of the second count component.

According to a second embodiment hereof, the present disclosure provides a dry powder inhaler device including an actuator for operating a dispensing mechanism of the dry powder inhaler device, a first count wheel configured to be rotated by the dispensing mechanism, and a second count component disposed adjacent to the first count wheel. The first count wheel includes a single tooth extending radially outward from a circumferential surface or side thereof. The second count component includes a plurality of rack teeth on an outer surface or side thereof, the plurality of rack teeth longitudinally aligned and extending outwards in a direction towards the single tooth of the first count wheel. The single tooth of the first count wheel engages one of the plurality of rack teeth of the second count component once per complete revolution of the first count wheel to intermittently translate the second count component. The first count wheel and the second count component collectively display a number of doses remaining within the inhaler device or a number of doses delivered by the inhaler device.

In an aspect of the second embodiment, and in combination with any other aspects herein, the disclosure provides that the actuator is a mouthpiece cover.

In an aspect of the second embodiment, and in combination with any other aspects herein, the disclosure provides that the plurality of rack teeth includes four rack teeth.

In an aspect of the second embodiment, and in combination with any other aspects herein, the disclosure provides that the first count wheel rotates about a first axis of the first count wheel and the second count component axially translates along a longitudinally-extending axis of the inhaler device.

In an aspect of the second embodiment, and in combination with any other aspects herein, the disclosure provides that the longitudinally-extending axis of the inhaler device is laterally spaced apart from the first axis.

In an aspect of the second embodiment, and in combination with any other aspects herein, the disclosure provides that the first count wheel is an annular component.

In an aspect of the second embodiment, and in combination with any other aspects herein, the disclosure provides that the second count component is a non-annular component.

In an aspect of the second embodiment, and in combination with any other aspects herein, the disclosure provides that an indicia-displaying surface of the first count wheel includes ‘ones’ digits disposed thereon and an indicia displaying surface of the second count component includes ‘tens’ digits disposed thereon.

In an aspect of the second embodiment, and in combination with any other aspects herein, the disclosure provides that the indicia-displaying surfaces of the first count wheel and the second count component are not coplanar, with the indicia displaying surface of the second count component disposed closer to a display window of the inhaler device than the indicia displaying surface of the first count wheel.

In an aspect of the second embodiment, and in combination with any other aspects herein, the disclosure provides that the second count component includes a single flag in place of a zero digit, the single flag being a colored block with no digits thereon.

In an aspect of the second embodiment, and in combination with any other aspects herein, the disclosure provides that the second count component includes a double flag, the double flag being a colored block with no digits thereon. The double flag is configured to cover a units digit of the first count wheel when no doses remain within the inhaler device.

In an aspect of the second embodiment, and in combination with any other aspects herein, the disclosure provides that the second count component is stationary when the single tooth is not engaged within one of the plurality of rack teeth.

In an aspect of the second embodiment, and in combination with any other aspects herein, the disclosure provides that the second count component includes a plurality of notches.

In an aspect of the second embodiment, and in combination with any other aspects herein, the disclosure provides that the first count wheel and the second count component are disposed within a housing of the dry powder inhaler device. The housing includes a flexible arm extending from an inner surface of the housing, the flexible arm including a detent thereon, the detent being configured to be received within a notch of the plurality of notches of the second count component.

In an aspect of the second embodiment, and in combination with any other aspects herein, the disclosure provides that the outer surface or side of the second count component including the plurality of rack teeth is a first outer surface or side, and the plurality of notches are disposed on a second outer surface or side opposing the first outer surface or side.

In an aspect of the second embodiment, and in combination with any other aspects herein, the disclosure provides that the circumferential surface or side of the first count wheel is an outer circumferential surface or side.

In an aspect of the second embodiment, and in combination with any other aspects herein, the disclosure provides that the circumferential surface or side of the first count wheel is an inner circumferential surface or side.

According to a third embodiment hereof, the present disclosure provides a dry powder inhaler device including an actuator for operating a dispensing mechanism of the dry powder inhaler device, a first count wheel configured to be rotated by the dispensing mechanism, and a second count component disposed at least partially within the first count wheel. The first count wheel includes a single tooth extending radially inward from an inner circumferential surface or side thereof. The second count component includes a plurality of gear teeth on an outer circumferential surface or side, each gear tooth extending radially outward and the second count component being non-concentric with the first count wheel. The single tooth of the first count wheel indirectly or directly engages one of the plurality of gear teeth of the second count component once per revolution of the first count wheel to intermittently rotate the second count component. The first count wheel and the second count component collectively display a number of doses remaining within the inhaler device or a number of doses delivered by the inhaler device.

In an aspect of the third embodiment, and in combination with any other aspects herein, the disclosure provides that the actuator is a mouthpiece cover.

In an aspect of the third embodiment, and in combination with any other aspects herein, the disclosure provides that the first count wheel rotates about a first axis and the second count component rotates about a second axis, the second axis being parallel to and spaced apart from the first axis.

In an aspect of the third embodiment, and in combination with any other aspects herein, the disclosure provides that the first count wheel is an annular component.

In an aspect of the third embodiment, and in combination with any other aspects herein, the disclosure provides that an indicia-displaying surface of the first count wheel includes ‘ones’ digits disposed thereon and an indicia displaying surface of the second count component includes ‘tens’ digits disposed thereon.

In an aspect of the third embodiment, and in combination with any other aspects herein, the disclosure provides that the indicia-displaying surfaces of the first count wheel and the second count component are not coplanar, with the indicia displaying surface of the second count component disposed closer to a display window of the inhaler device than the indicia displaying surface of the first count wheel.

In an aspect of the third embodiment, and in combination with any other aspects herein, the disclosure provides that the second count component includes a single flag in place of a zero digit, the single flag being a colored block with no digits thereon.

In an aspect of the third embodiment, and in combination with any other aspects herein, the disclosure provides that the second count component includes a double flag, the double flag being a colored block with no digits thereon. The double flag is configured to cover a units digit of the first count wheel when no doses remain within the inhaler device.

In an aspect of the third embodiment, and in combination with any other aspects herein, the disclosure provides that the second count component is stationary when the single tooth is not engaged with one of the plurality of gear teeth of the second count component.

In an aspect of the third embodiment, and in combination with any other aspects herein, the disclosure provides that the single tooth of the first count wheel directly engages the plurality of gear teeth of the second count component. The first count wheel and the second count component are configured to rotate in the same direction.

In an aspect of the third embodiment, and in combination with any other aspects herein, the disclosure provides the dry powder inhaler device further includes an intermediate gear disposed between the inner circumferential surface or side of the first count wheel and the outer circumferential surface or side of the second count component. The single tooth of the first count wheel directly engages the intermediate gear and the intermediate gear directly engages the plurality of gear teeth of the second count component. The first count wheel and the second count component are configured to rotate in opposing directions.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1A is a front view of an inhaler device according to an embodiment hereof, wherein a mouthpiece cover of the inhaler device is in a closed position.

FIG. 1B is a rear view of the inhaler device of FIG. 1A, wherein the mouthpiece cover of the inhaler device is in the closed position.

FIG. 1C is a front view of the inhaler device of FIG. 1A, wherein the mouthpiece cover of the inhaler device is in an open position.

FIG. 1D is a graph that illustrates an estimated actuation force profile for the inhaler device and mouthpiece cover as depicted in FIGS. 1A-1C (in the dotted line), compared to an estimated actuation force profile for an inhaler device in which a dispensing mechanism is not actuated for an initial period of travel of a mouthpiece cover (in the solid line).

FIG. 2 is a perspective view of two blister strips for use within the inhaler device of FIG. 1A.

FIG. 3A is a front view of the inhaler device of FIG. 1A, wherein the mouthpiece cover of the inhaler device is in the open position and a housing of the inhaler device is removed for sake of illustration only.

FIG. 3B is a sectional perspective view of a portion of the inhaler device of FIG. 1A illustrating a portion of the airflow path through the inhaler device of FIG. 1A.

FIG. 4 is a perspective view of a manifold of the inhaler device of FIG. 1A, wherein the manifold is removed from the inhaler device for sake of illustration only.

FIG. 4A is a sectional view taken along line A-A of FIG. 4.

FIG. 5 is a perspective view of the manifold of FIG. 4 positioned adjacent to inlet vents of the housing of the inhaler device of FIG. 1A.

FIG. 6 is another perspective view of a manifold of the inhaler device of FIG. 1A, wherein the manifold is removed from the inhaler device for sake of illustration only.

FIG. 7 is a schematic view illustrating an airflow path through the manifold of FIG. 6.

FIG. 8 is a schematic flowchart illustrating the airflow path through the manifold of FIG. 6.

FIG. 9A is a front view of the inhaler device of FIG. 1A, wherein the mouthpiece cover of the inhaler device is in the open position and a portion of the housing of the inhaler device is removed for sake of illustration only.

FIG. 9B is a rear view of the inhaler device of FIG. 1A, wherein the mouthpiece cover and the housing of the inhaler device is removed for sake of illustration only.

FIG. 10 is a front view of a ratchet mechanism of the inhaler device of FIG. 1A, wherein the ratchet gear is removed from the inhaler device for sake of illustration only.

FIG. 10A illustrates the ratchet mechanism of FIG. 10 when the mouthpiece cover is in the closed position.

FIG. 10B illustrates the ratchet mechanism of FIG. 10 when the mouthpiece cover is in the open position.

FIG. 11 is a perspective view of the mouthpiece cover and a portion of a dispensing subassembly of the inhaler device of FIG. 1A, wherein the mouthpiece cover and dispensing subassembly are removed from the inhaler device for sake of illustration only.

FIG. 12A is a schematic illustration of a tensioning mechanism of the inhalation device of FIG. 1A, wherein the tensioning mechanism is shown at a beginning of the device life.

FIG. 12B is a schematic illustration of a tensioning mechanism of the inhalation device of FIG. 1A, wherein the tensioning mechanism is shown near an end of the device life.

FIG. 13A is a perspective view of a tensioning mechanism of the inhalation device of FIG. 1A, wherein the tensioning mechanism is removed from the inhalation device for sake of illustration only.

FIG. 13B is a sectional view of the tensioning mechanism of FIG. 13A.

FIG. 13C is a cross-sectional view of the tensioning mechanism of FIG. 13A.

FIG. 13D is a perspective exploded view of a base and a nut of the tensioning mechanism of FIG. 13A.

FIG. 13E is a series of sectional views of the tensioning mechanism of FIG. 13A illustrating the movement of the nut during operation of the inhalation device.

FIG. 14A is a perspective view of a counter subassembly of the inhaler device of FIG. 1A, wherein the counter subassembly is removed from the inhaler device for sake of illustration only.

FIG. 14B is a front view of the counter subassembly of FIG. 14A.

FIG. 14C is a front sectional view of the counter subassembly of FIG. 14A, taken along line C-C of FIG. 14B.

FIG. 14D is a front sectional view of the counter subassembly of FIG. 14A, taken along line D-D of FIG. 14C.

FIG. 15A is a perspective view of a counter subassembly according to another embodiment hereof, wherein the counter subassembly is configured for use in the inhaler device of FIG. 1A, and wherein the counter subassembly displays twenty-one doses remaining.

FIG. 15B is another perspective view of the counter subassembly of FIG. 15A, wherein the counter subassembly displays zero doses remaining.

FIG. 16A is a front view of a counter subassembly according to another embodiment hereof, wherein the counter subassembly is configured for use in the inhaler device of FIG. 1A, and wherein the counter subassembly displays twenty-six doses remaining.

FIG. 16B is a front sectional view of the counter subassembly of FIG. 16A, taken along line B-B of FIG. 16A.

FIG. 17 is a front view of a counter subassembly according to another embodiment hereof, wherein the counter subassembly is configured for use in the inhaler device of FIG. 1A, and wherein the counter subassembly displays twenty-six doses remaining.

FIG. 18A is a front view of a counter subassembly according to another embodiment hereof, wherein the counter subassembly is configured for use in the inhaler device of FIG. 1A, and wherein the counter subassembly displays twenty-seven doses remaining.

FIG. 18B is a front sectional view of the counter subassembly of FIG. 18A, taken along line B-B of FIG. 18A.

FIG. 19A is a front view of a counter subassembly according to another embodiment hereof, wherein the counter subassembly is configured for use in the inhaler device of FIG. 1A, and wherein the counter subassembly displays twenty-six doses remaining.

FIG. 19B is a front sectional view of the counter subassembly of FIG. 19A, taken along line B-B of FIG. 19A.

FIG. 20A is a front view of a counter subassembly according to another embodiment hereof, wherein the counter subassembly is configured for use in the inhaler device of FIG. 1A, and wherein the counter subassembly displays thirty doses remaining.

FIG. 20B is a front exploded view of the counter subassembly of FIG. 20A.

FIG. 20C is a front view of a first count wheel, a second count wheel, and a transfer gear of the counter subassembly of FIG. 20A.

FIG. 20D is a partial perspective view of the counter subassembly of FIG. 20A.

FIG. 20E is a schematic cross-sectional view of a lens of the counter subassembly of FIG. 20A.

FIG. 20F is a partial perspective view of the counter subassembly of FIG. 20A, along with a first bottom sheet take up gear for use in the inhaler device of FIG. 1A.

FIG. 20G is an enlarged perspective view of the first bottom sheet take up gear of FIG. 20F.

FIG. 20H is an enlarged perspective bottom view of the transfer gear of FIG. 20F.

FIG. 20I is a front stepped sectional view of the counter subassembly of FIG. 20A.

FIG. 20J is a front view of a back plate of the counter subassembly of FIG. 20A.

FIG. 20K is a back view of the first count wheel of the counter subassembly of FIG. 20A.

FIG. 20L is a sectional view of the first count wheel of the counter subassembly of FIG. 20A disposed within the back plate of FIG. 20J, wherein a first flexible arm is in a first position.

FIG. 20M is a sectional view of the first count wheel of the counter subassembly of FIG. 20A disposed within the back plate of FIG. 20J, wherein a first flexible arm is in a second position.

### DETAILED DESCRIPTION OF EMBODIMENTS OF THE INVENTION

Specific embodiments of the present invention are now described with reference to the figures, wherein like reference numbers indicate identical or functionally similar elements. The following detailed description is merely exemplary in nature and is not intended to limit the invention or the application and uses of the invention. Although the description of the invention is in the context of dry powder inhaler devices, the invention may also be used in other applications where it is deemed useful. Furthermore, there is no intention to be bound by any expressed or implied theory presented in the preceding technical field, background, brief summary or the following detailed description.

Embodiments hereof relate to inhaler devices configured to deliver powdered medicament from at least one blister strip, and more particularly, relate to counter mechanisms for displaying the number of doses remaining within the inhaler device. The counter mechanisms described herein are illustrated within an inhaler device configured to simultaneously deliver powdered medicament from two blister strips but may also be utilized in an inhaler device configured to deliver powdered medicament from a single blister strip or more than two blister strips.

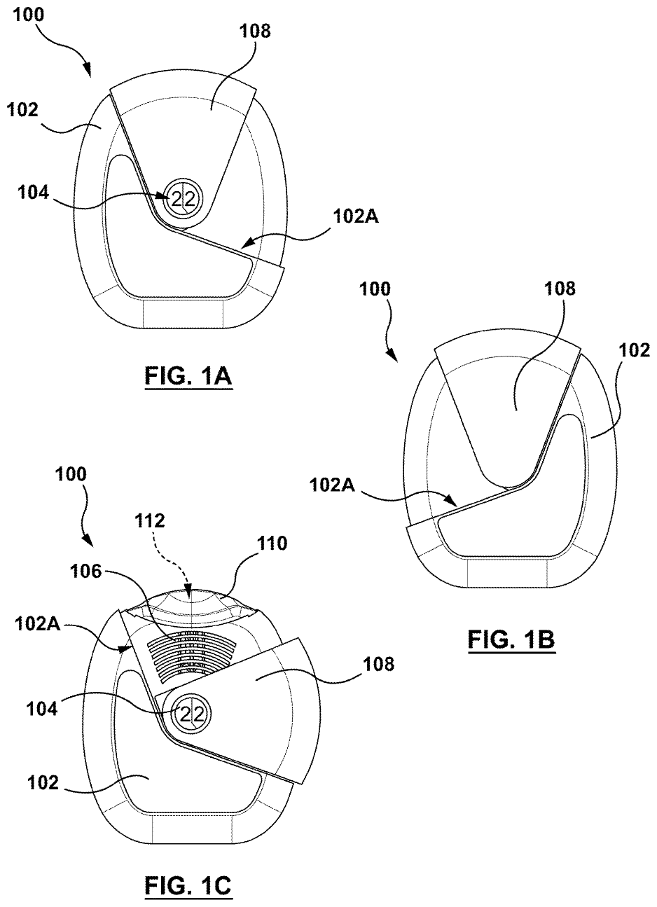

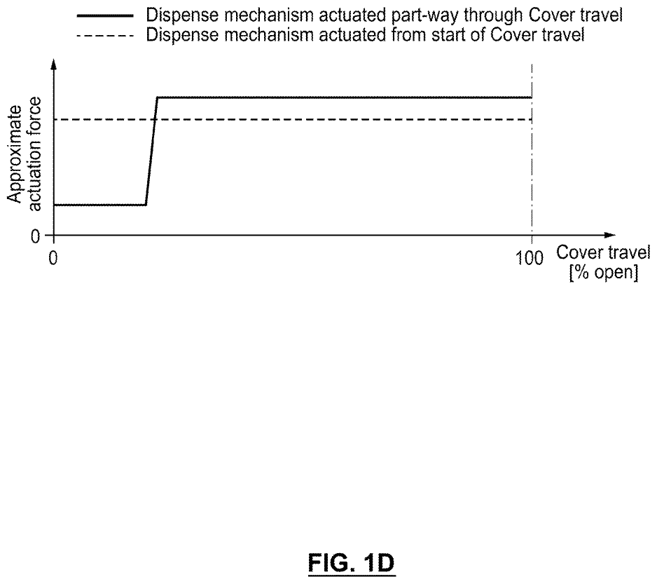

FIGS. 1A, 1B, and 1C illustrate an inhaler device 100 according to an embodiment hereof. The inhaler device 100 includes a housing 102 and a mouthpiece cover 108. The housing 102 includes a display window 104 through which a number is displayed, the number indicating the number of remaining doses of the inhaler device 100. The housing 102 also includes a plurality of openings or inlet vents 106 formed through a sidewall of the housing 102. As will be described in more detail herein, air from outside of the inhaler device 100 is drawn into the interior of the inhaler device 100 via the inlet vents 106 upon inhalation, by a user, at a mouthpiece 110. In FIGS. 1A and 1B, the mouthpiece cover 108 of the inhaler device 100 is in a closed position in which the mouthpiece cover 108 covers or extends over the mouthpiece 110. The mouthpiece cover 108 is in an open position in FIG. 1C such that the mouthpiece 110 is exposed and available to the user. The user can inhale the powdered medicament through the mouthpiece 110 only when the mouthpiece cover 108 is in the open configuration. The mouthpiece 110 includes a central outlet or opening 112 which permits delivery of the powdered medicament contained within the inhaler device 100 to the user via inhalation.

In the closed position of FIGS. 1A and 1B, the mouthpiece 110 and the inlet vents 106 are covered by the mouthpiece cover 108. When a user wishes to inhale a medicament dose from the inhaler device 100, the mouthpiece cover 108 is moved from the closed position of FIGS. 1A and 1B to the open position of FIG. 1C. In the open position, the mouthpiece cover 108 is rotated or moved relative to the housing 102 such that the mouthpiece 110 and the inlet vents 106 are fully exposed and no longer covered by any portion of the mouthpiece cover 108. Since users are instructed to cover the central opening 112 of the mouthpiece 110 with their mouth during inhalation of the powdered medicament, the mouthpiece cover 108 protects the mouthpiece 110 when the inhaler device 100 is not in use to prevent contamination of the airflow passageway of the inhaler device 100 by unwanted particles, which could otherwise have a negative impact on user experience and/or dose delivery. As will be described in more detail herein, the movement of the mouthpiece cover 108 from the closed position to the open position actuates a dispensing mechanism in the inhaler device 100 to make a medicament dose available for inhalation and further actuates a counter mechanism in the inhaler device 100 to decrease the number of remaining doses shown in the display window 104 by one unit. Thus, the mouthpiece cover 108 functions to protect the central opening 112 of the mouthpiece 110 and also operates the dispensing and counter mechanisms of the inhaler device 100. Only a single operating step, i.e., movement of the mouthpiece cover 108, is required by the user to actuate the inhaler device 100 for each dose.

In an embodiment, a mouthpiece cover 108 may be rotated between 85 and 105 degrees by the user to expose a mouthpiece 110 and inlet vents 106. In designing the inhaler device 100, it is important to ensure that a force required to actuate the mouthpiece cover 108 is sufficiently low to allow easy operation by users with a range of abilities. In general, when a distance of travel of the mouthpiece cover 108 is longer, a more favorable mechanical advantage is realized in a dispensing mechanism of the inhaler device, which results in a lower actuation force being needed to rotate the mouthpiece cover 108 and actuate the inhaler device 100 for each dose. However, from an ergonomics perspective, a shorter distance of travel of the mouthpiece cover 108 may avoid the user having to change grip during actuation. A shorter travel distance of the mouthpiece cover 108 also results in a larger area of the housing 102 for the user to grip during actuation, since there is a smaller area of the housing 102 traversed by the mouthpiece cover 108. A shorter travel of the mouthpiece cover 108 also allows more space for other features of the inhaler device 100, and/or allows a size of the inhaler device 100 to be minimized. In an embodiment, a cover travel between 90 and 100 degrees may provide an optimal balance between the above noted factors. As shown in FIGS. 1A-1C, the housing 102 includes an integral flange or step 102A formed thereon that controls or limits rotational movement of the mouthpiece cover 108 to the desired range.

The force profile over the travel of the mouthpiece cover 108 will affect the user experience and tactile feedback given by the inhaler device 100. Maintaining a relatively consistent or constant actuation force over the travel of the mouthpiece cover 108 is also preferable to avoid incorrect usage or confusion. Using the full travel of the mouthpiece cover 108 for operating the dispensing mechanism is expected to make the actuation force profile more consistent and help to mitigate the risk of misuse of the inhaler device 100. For example, in inhaler devices unlike the present invention in which a dispensing mechanism is not actuated for an initial period of the mouthpiece cover travel, an actuation force is relatively low in this initial period. At the point in which the dispensing mechanism is actuated, the actuation force of the mouthpiece cover increases. Thus in inhalers unlike the present invention, the actuation force of the mouthpiece cover significantly increases midway through the total travel of the mouthpiece cover, and a user may incorrectly perceive this change as tactile feedback suggesting that the mouthpiece cover is sufficiently open to take a dose. Stated another way, a non-constant actuation force profile may be confusing for a user in terms of the tactile feedback and could cause incorrect usage of the device. FIG. 1D illustrates an estimated actuation force profile, represented by a dashed line, for the inhaler device 100 according to embodiments herein that has the mouthpiece cover 108, as compared to an estimated actuation force profile, represented by a solid line, for an inhaler device in which the dispensing mechanism is not actuated for an initial period of the mouthpiece cover travel as explained in the prior example. When a dispensing mechanism does not actuate at the beginning of the mouthpiece travel, there is a step in the actuation force profile as represented by the solid line in FIG. 1D. Conversely, when a dispensing mechanism actuates at the beginning of the mouthpiece travel as in the inhaler device 100, then the actuation force is generally constant or consistent and the peak actuation force is lower, as represented by the dashed line in FIG. 1D.

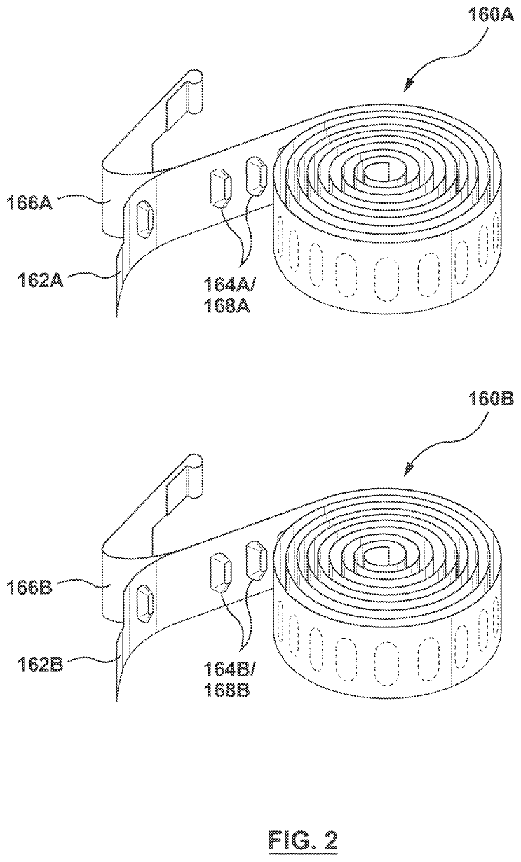

Inhaler device 100 is configured to simultaneously dispense dry powder medicaments from two blister strips. More particularly, with reference to FIG. 2, a first blister strip 160A and a second blister strip 160B are shown. Inhaler device 100 described herein is configured to simultaneously dispense medicament from each of the first and second blister strips 160A, 160B. Each blister strip 160A, 160B includes a bottom sheet 162A, 162B, respectively, which defines a series or plurality of individual blisters or pockets 164A, 164B thereon. Each pocket 164A, 164B is configured to contain a dose or portion thereof of dry powder or powdered medicaments 168A, 168B to be inhaled by a user. In an embodiment, the powdered medicament 168A is a different medicament than the powdered medicament 168B so that the inhaler device 100 is configured to simultaneously deliver two different powdered medicaments to the user. A top sheet 166A, 166B is hermetically bonded or sealed to the bottom sheet 162A, 162B, respectively, to close the pockets 164A, 164B, respectively and function as a lid for the pockets 164A, 164B to retain the powdered medicaments 168A, 168B therein. The hermetic sealing of the top sheets 166A, 166B is such that the bottom sheets 162A, 162B and the top sheets 166A, 166B are able to be peeled apart to open or uncover the pockets 164A, 164B for access to the powdered medicaments 168A, 168B therein. Each of the first and second blister strips 160A, 160B is sufficiently flexible to be wound into a roll.

As will be described in more detail herein, when a dispensing mechanism of the inhaler device 100 is actuated via movement of the mouthpiece cover 108, the top sheets 166A, 166B of the blister strips 160A, 160B, respectively, are peeled away from the bottom sheets 162A, 162B of the blister strips 160A, 160B, respectively, to open or uncover a pocket 164A, 164B of each blister strip and thereby expose the powdered medicaments 168A, 168B disposed therein. Upon inhalation via the mouthpiece 110, the user simultaneously inhales the powdered medicaments 168A, 168B from the opened pockets 164A, 164B of the blister strips 160A, 160B, respectively. The user thus receives a fixed metered dose of medicament powder of which the different medicament powders from the opened pockets 164A, 164B of the blister strips 160A, 160B, respectively, make up respective dose portions. Each blister strip 160A, 160B may be of the same size and/or contain the same dose amount (e.g., volume or mass) of powdered medicament or may be of different sizes and/or contain different dose amounts of powdered medicament.

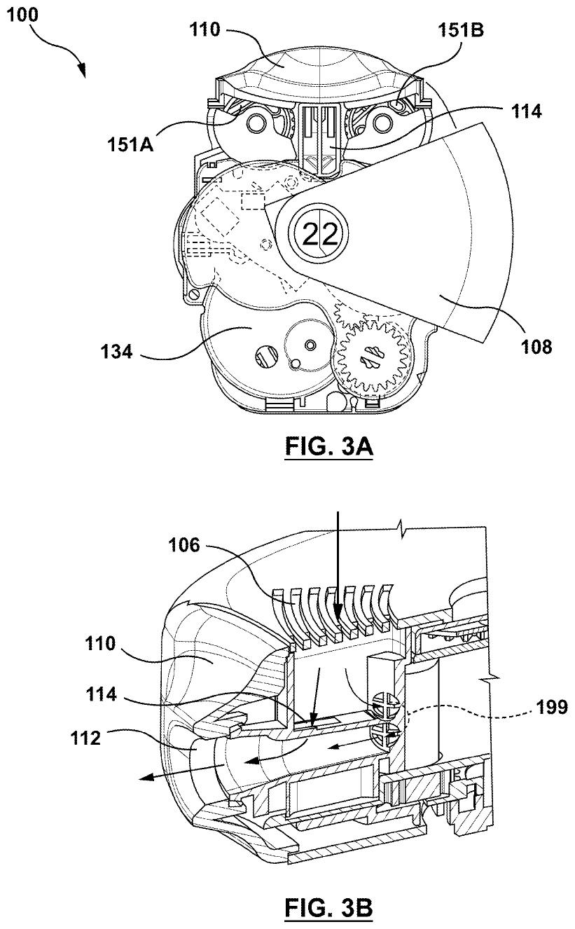

FIG. 3A is a front view of the inhaler device 100 with the mouthpiece cover 108 in the open position and the housing 102 removed for sake of illustration only. The inhaler device 100 includes a manifold 114 for directing airflow therethrough to entrain and deliver the powdered medicaments 168A, 168B from the blister strips 160A, 160B, respectively, to the user via the mouthpiece 110. The manifold 114 is in fluid communication with the mouthpiece 110 such that the powdered medicaments 168A, 168B may be delivered to the user through the central opening 112 of the mouthpiece 110. In addition to the manifold 114, the inhaler device 100 also includes a dispensing subassembly or mechanism 120, a counter subassembly or mechanism 134, and tensioning subassemblies or mechanisms 151A, 151B. When assembled, each of the manifold 114, the dispensing subassembly 120, the counter subassembly 134, and the tensioning mechanisms 151A, 151B reside or are disposed within the housing 102.

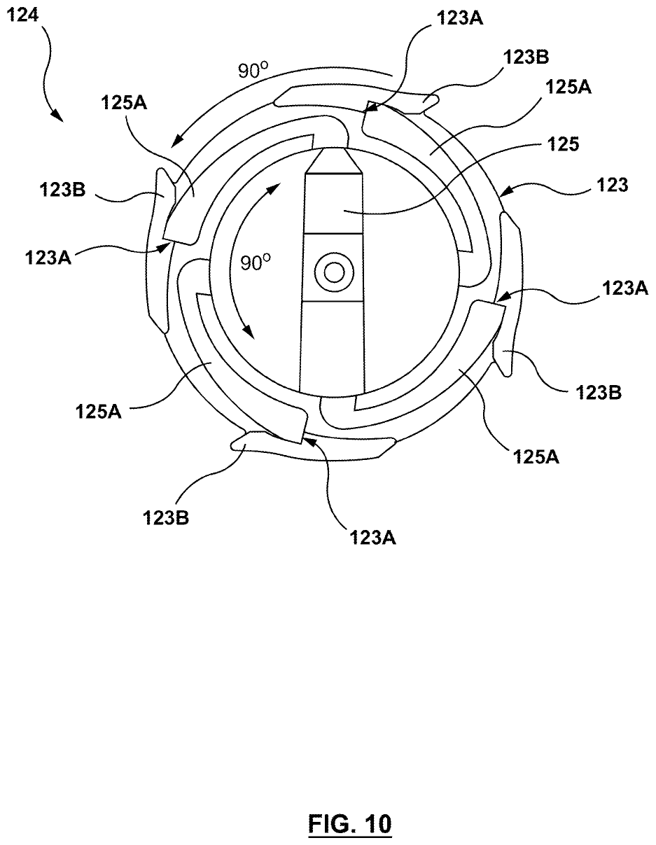

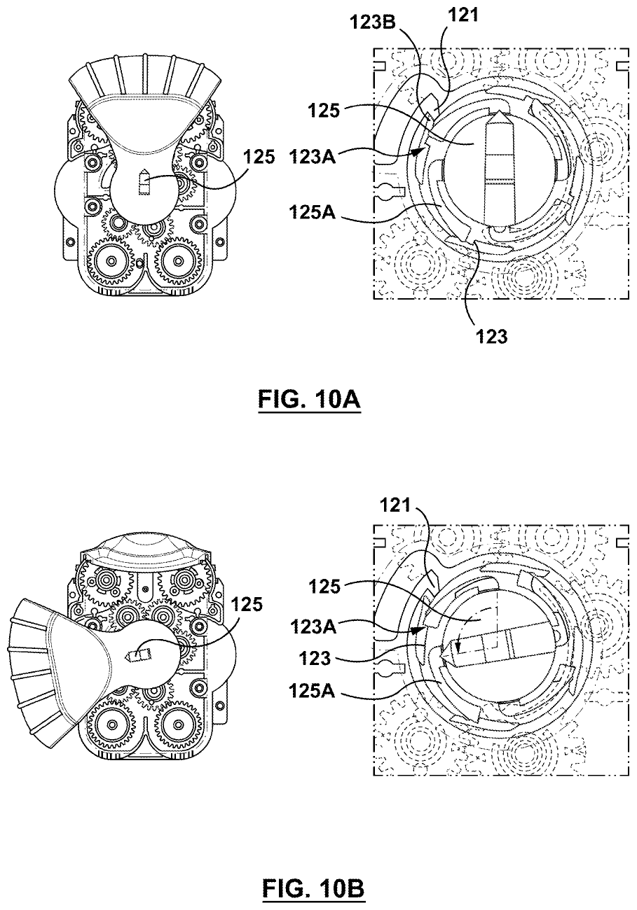

The operation of the manifold 114 is introduced herein with reference to FIG. 3B. The manifold 114 defines the air pathway through the inhaler device 100. The manifold 114 fluidly connects the mouthpiece 110 to the first and second blister strips 160A, 160B. FIG. 3B is a sectional view taken through the manifold 114 to illustrate an airflow path therethrough for entrainment of medicament 168B of the second blister strip 160B. As explained with respect to FIGS. 10-14, the manifold 114 also defines an airflow path therethrough for entrainment of medicament 168A of the first blister strip 160A.

In use, the mouthpiece cover 108 is rotated by the user to expose the mouthpiece 110 and the inlet vents 106. Internally, within the inhaler device 100, rotating the mouthpiece cover 108 exposes the powdered medicaments 168A, 168B within a pocket 164A, 164B, respectively, of each of first and second blister strips 160A, 160B. To access the powdered medicaments 168A, 168B within the opened pockets 164A, 164B, the user breathes in or inhales through the mouthpiece 110. By covering the central opening 112 of the mouthpiece 110 with their mouth and inhaling, the user creates a pressure differential between the inlet vents 106 and the central opening 112 and causes air to travel through the manifold 114. The pressure differential causes external air (i.e., air from outside of the inhaler device 100) to enter the inhaler device 100 through the inlet vents 106, pass through the opened pockets 164A, 164B, and exit the inhaler device 100 through the central opening 112. The airflow pathways defined by the manifold 114 are designed such that when the user inhales, the exposed powdered medicaments 168A, 168B within the opened pockets 164A, 164B are picked up by the airstreams and delivered to the user as an orally inhaled combined medicament dose. As such, the user can simultaneously inhale one dose portion from each blister strip 160A, 160B.

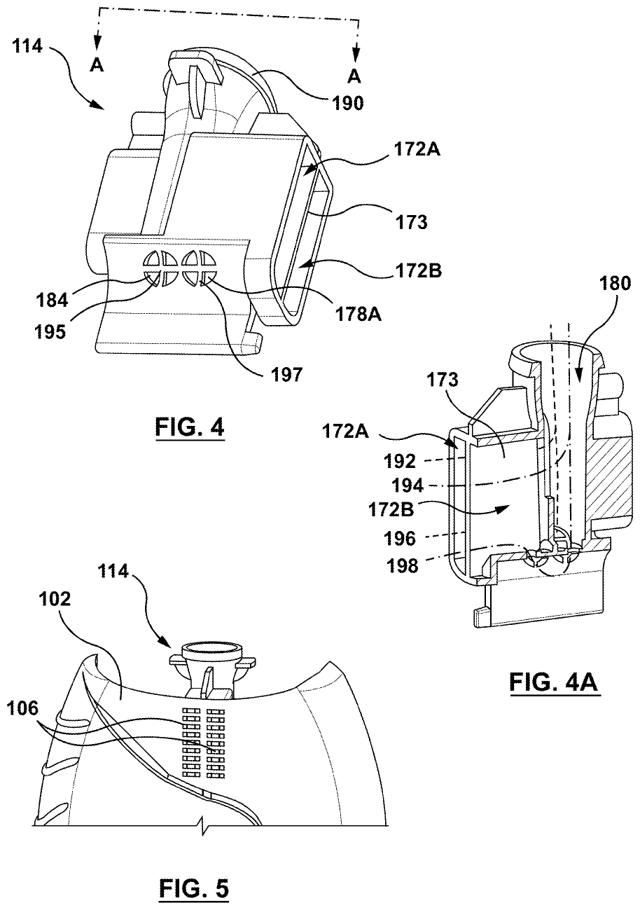

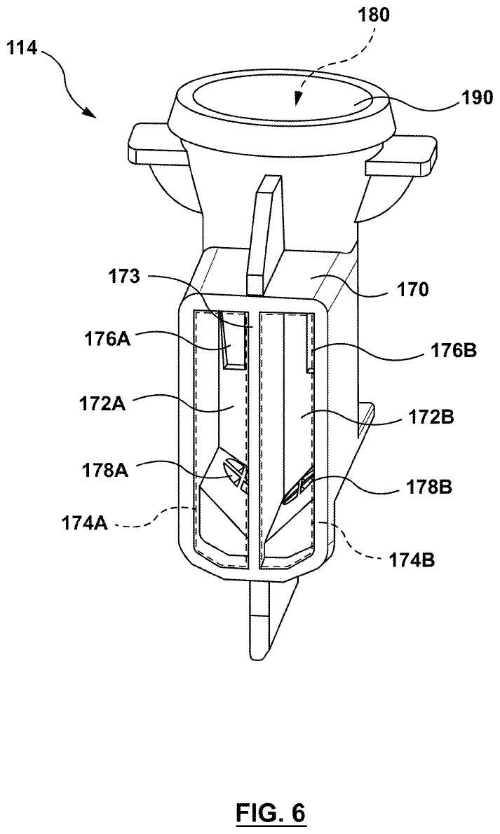

The manifold 114 is configured to preferentially direct the inhalation airflow in a variety of ways to achieve airflow properties that are advantageous for effective delivery of the powdered medicaments. More particularly, as shown in FIG. 3B, the geometry of manifold 114 causes a portion of the inhalation airflow to enter and exit an opened pocket 164B of the second blister strip 160B (labeled with a dotted line 199 in FIG. 3B), while another portion of the inhalation airflow travels through a diversion or hole in the manifold 114 (which is described in more detail in FIGS. 4-8). The portion of inhalation airflow through the opened pocket 164B results in entrainment of the dose of powdered medicament 168B in the airflow, and the portion of inhalation airflow through the diversion in the manifold 114 intersects the entrained airflow portion to break up the powdered medicament therein before exiting the manifold 114.

Turning to FIGS. 4-8, the manifold 114 is described in more detail. The manifold 114 is configured for simultaneous delivery of powdered medicament 168A, 168B from respective open blister pockets 164A, 164B, of each of the first blister strip 160A and the second blister strip 160B, respectively. The manifold 114 includes a body 170 that defines a first space or atrium 172A, a second space or atrium 172B, and a stack 180. As will be explained in more detail herein, the separated distinct compartments or spaces of the first atrium 172A, the second atrium 172B, and the stack 180 split up, divide, or otherwise separate an inhalation airflow, which is drawn into the manifold 114 by a user, into multiple airflow paths through the body 170 of the manifold 114. More specifically, when an inhalation force is applied through the central opening 112 of the mouthpiece 110, an inhalation airflow is drawn into the first and second atriums 172A, 172B of the manifold 114 via the inlet vents 106 of the inhaler device 100. The first and second atriums 172A, 172B are disposed adjacent to, or are in a juxtaposed relation with, the inlet vents 106. Once the inhalation airflow enters into the manifold 1114, it splits or divides into four airflow paths as it travels through the body 170 of the manifold, namely, a first diversion airflow path 192, a second diversion airflow path 194, a first entrainment airflow path 196, and a second entrainment airflow path 198. Stated another way, each of the first diversion airflow path 192, the second diversion airflow path 194, the first entrainment airflow path 196, and the second entrainment airflow path 198 are respective airflow portions of the inhalation airflow which is drawn into the manifold 114.

In this embodiment, the first atrium 172A and the second atrium 172B are disposed laterally adjacent to each other, or side-by-side, on a single side of the body 170 of the manifold 114. The first atrium 172A and the second atrium 172B are separated from each other by a divider wall 173 such that the first atrium 172A is not in fluid communication with the second atrium 172B. The first atrium 172A includes a single atrium inlet 174A, and the second atrium 172B includes a single atrium inlet 174B. The atrium inlets 174A, 174B are separate from each other and may also be considered the inlets of the manifold 114. As such, the manifold 114 includes two inlets, the atrium inlet 174A leading or entering into the first atrium 172A and the atrium inlet 174B leading or entering into the second atrium 172B.

The first atrium 172A includes a first atrium outlet 176A and a second atrium outlet 178A, and the second atrium 172B includes a first atrium outlet 176B and a second atrium outlet 178B. As will be explained in more detail herein, the first atrium outlet 176A, 176B of each of the first and second atriums 172A, 172B, respectively, directs or guides flow directly into the stack 180 and the second atrium outlet 178A, 178B of each of the first and second atriums 172A, 172B, respectively, directs or guides flow into an open pocket 164A, 164B, respectively, of the first and second blister strips 160A, 160B, respectively. In an embodiment, the profile or shape of the first atrium outlet 176A, 176B is substantially rectangular or oblong. However, the profile or shape of the first atrium outlets 176A, 176B is not limited to the shape depicted herein and may alternatively be circular, triangular, or any other shape deemed suitable for the purposes described herein. Similarly, the profile or shape of the second atrium outlets 178A, 178B is substantially circular and includes a grill or cross-piece 197 (see FIG. 4) spanning thereover to encourage increased turbulence in the airflow. However, the profile or shape of the second atrium outlets 178A, 178B is not limited to the shape depicted herein and may alternatively be rectangular, oblong, oval, triangular, or any other shape deemed suitable for the purposes described herein and may or may not include a grill spanning thereover.

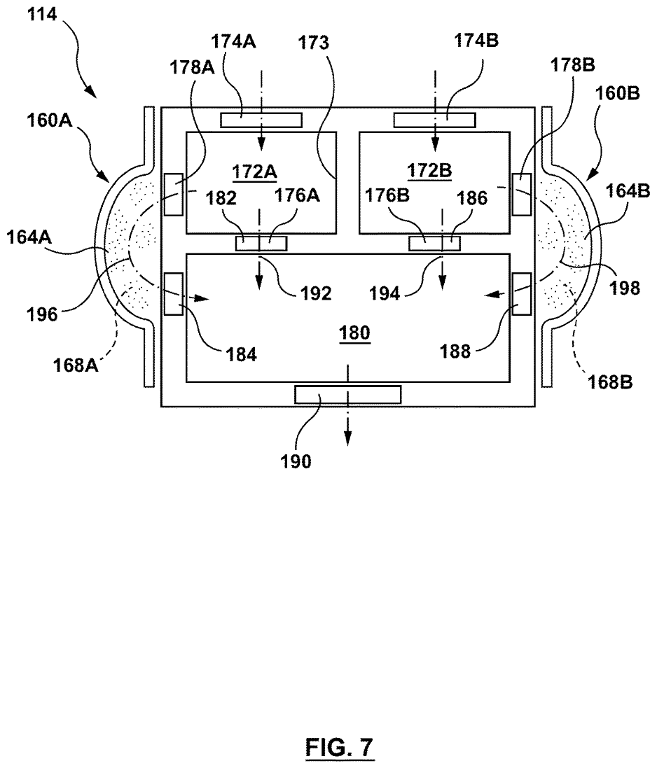

As best shown on FIG. 7, the stack 180 is in fluid communication with each of the first atrium 172A, the second atrium 172B, the open pocket 164A of the first blister strip 160A, and the second open pocket 164B of the second blister strip 160B. The stack 180 has four inlets, namely, a first stack inlet 182, a second stack inlet 184, a third stack inlet 186, and a fourth stack inlet 188. The first stack inlet 182 is aligned with the first atrium outlet 176A of the first atrium 172A, such that the stack 180 and the first atrium 172A are in fluid communication with each other. The second stack inlet 184 is aligned with the open pocket 164A of the first blister strip 160A, such that the second stack inlet 184 is further in fluid communication with the second atrium outlet 178A of the first atrium 172A via the open pocket 164A. The third stack inlet 186 is in fluid communication with the second atrium outlet 176B of the second atrium 172B, such that the stack 180 and the second atrium 172B are in fluid communication with each other. The fourth stack inlet 188 is in fluid communication with the open pocket 164B of the second blister strip 160B, such that the fourth stack inlet 188 is further in fluid communication with the second atrium outlet 178B of the second atrium 172B via the open pocket 164B.

The profile or shape of each of the first stack inlet 182 and the third stack inlet 186 is substantially rectangular or oblong. However, the profile or shape of the first and third stack inlets 182, 186 is not limited to the shape depicted herein and may alternatively be circular, triangular, or any other shape deemed suitable for the purposes described herein. Similarly, the profile or shape of the second stack inlet 184 and the fourth stack inlet 188 is substantially circular and includes a grill or cross-piece 195 (see FIG. 4) spanning thereover to encourage increased turbulence in the airflow. However, the profile or shape of the second and fourth stack inlets 184, 188 is not limited to the shape depicted herein and may alternatively be rectangular, oblong, oval, triangular, or any other shape deemed suitable for the purposes described herein and may or may not include a grill spanning thereover.

The stack 180 is a single stack with a single stack outlet 190. Thus in the present embodiment, the stack 180 has only one stack outlet 190. The stack outlet 190 may also be considered the outlet of the manifold 114. As such, the manifold 114 includes only one outlet. A shape or profile of the stack outlet 190 is oval. However, the profile or shape of the stack outlet 190 is not limited to the shape depicted herein and may alternatively be circular, rectangular, oblong, triangular, or any other shape deemed suitable for the purposes described herein. When the manifold 114 is assembled into the inhaler device 100, the stack outlet 190 is aligned and in fluid communication with the central opening 112 of the mouthpiece 110.

The second atrium outlet 178A of the first atrium 172A is in fluid communication with the second stack inlet 184 to define the first entrainment airflow path 196 associated with an open blister pocket 164A of the first blister strip 160A. As the airstream flows through the open blister pocket 164A, it picks up the powdered medicament 168A disposed within the open blister pocket 164A. Thus, by passing through the open blister pocket 164A, the powdered medicament 168A is drawn in and transported by the airstream into the stack 180. After entrainment, the airstream is laden with the powdered medicament 168A.

Similarly, the second atrium outlet 178B of the second atrium 172B is in fluid communication with the fourth stack inlet 188 to define the second entrainment airflow path 198 associated with an open blister pocket 164B of the second blister strip 160B. As the airstream flows through the open blister pocket 164B, it picks up the powdered medicament 168B disposed within the open blister pocket 164B. Thus, by passing through the open blister pocket 164B, the powdered medicament 168B is drawn in and transported by the airstream into the stack 180. After entrainment, the airstream is laden with the powdered medicament 168B.

The first atrium outlet 176A of the first atrium 172A is in fluid communication with the first stack inlet 182 to define the first diversion airflow path 192 of the manifold 114. Similarly, the first atrium outlet 176B of the second atrium 172B is in fluid communication with the third stack inlet 186 to define the second diversion airflow path 194 of the manifold 114. Each of the diversion airflow paths 192, 194 provides a lower resistance pathway for air to flow from outside the inhaler device 100 to the patient's mouth compared to the first and second entrainment airflow paths 196, 198. As a result, the overall airflow resistance of the inhaler device 100 is lowered so that higher overall flow rates can be achieved for the same inhalation pressure. In addition, the diversion airflow paths 192, 194 provide de-agglomeration of the powdered medicaments 168A, 168B before they exit the manifold 114. Each of the first and second diversion airflow paths 192, 194 is configured to disrupt each of the first and second entrainment airflow paths 196, 198 and break up medicament carried thereby. More particularly, the first diversion airflow path 192 is directed into the path of the first entrainment airflow path 196, which is at a different angle from the first diversion airflow path 192. A region of higher shear is created at the point of intersection between the first diversion airflow path 192 and the first entrainment airflow path 196, improving de-agglomeration of the powdered medicament 168A before it exits the manifold 114. Similarly, the second diversion airflow path 194 is directed into the path of the second entrainment airflow path 198, which is at a different angle from the second diversion airflow path 194. A region of higher shear is created at the point of intersection between the second diversion airflow path 194 and the second entrainment airflow path 198, improving de-agglomeration of the powdered medicament 168B before it exits the manifold 114. The first diversion airflow path 192, the second diversion airflow path 194, the first entrainment airflow path 196, and the second entrainment airflow path 198 combine or mix together within the stack 180 before exiting the manifold 114.

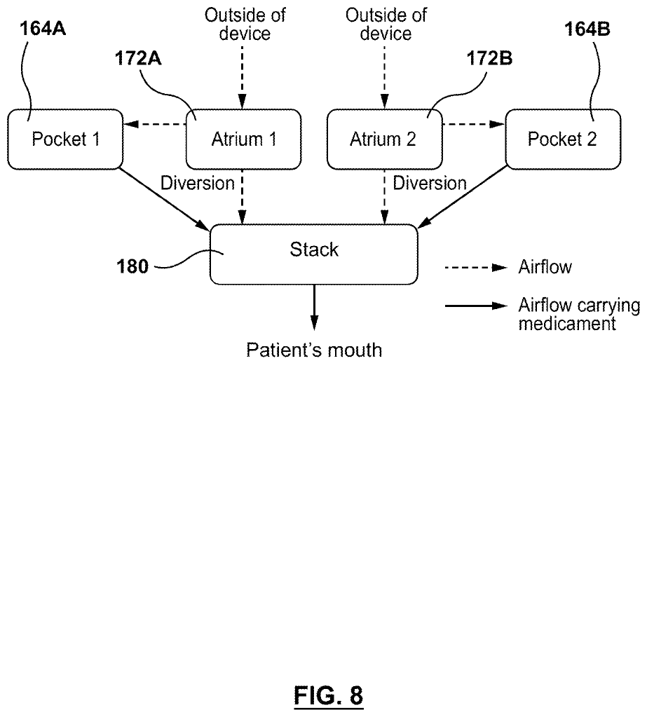

As represented in the flowchart of FIG. 8, the inhalation airstream drawn from outside the inhaler device 100 is divided between the two inlets of the manifold 114, namely the first atrium inlet 174A and the second atrium inlet 174B. The inhalation airstream drawn from outside the inhaler device thus simultaneously enters each of the first atrium 172A and the second atrium 172B. A first portion of the inhalation airstream entering the first atrium 172A flows into the open blister pocket 164A and a second portion of the inhalation airstream entering the first atrium 172A directly flows into the stack 180. The first portion of the inhalation airstream within the open blister pocket 164A picks up or entrains powdered medicament 168A disposed within the open blister pocket 164A, and then continues into the stack 180. Within the stack 180, the second portion of the inhalation airstream from the first atrium 172A breaks up or de-agglomerates the powdered medicament 168A entrained within the first portion of the inhalation airstream. Similarly, at the same time, a first portion of the inhalation airstream entering the second atrium 172B flows into the open blister pocket 164B and a second portion of the inhalation airstream entering the second atrium 172B flows into the stack 180. The first portion of the inhalation airstream within the open blister pocket 164B picks up or entrains powdered medicament 168B disposed within the open blister pocket 164B, and then continues into the stack 180. Within the stack 180, the second portion of the inhalation airstream from the second atrium 170B breaks up or de-agglomerates the powdered medicament 168B entrained within the first portion of the inhalation airstream. Within the stack 180, all portions of the airstream mix together before exiting the manifold 114 towards a patient's mouth, with the combined airstream including both medicament 168A from the first blister strip 160A and medicament 168B from the second blister strip 160B. Airflow through the first atrium 172A and the open blister pocket 164A is concurrent to airflow through the second atrium 172B and the open blister pocket 164B.

Due to the fact that the first and second atriums 172A, 172B are separate and distinct compartments with the divider wall 173 extending therebetween, the inhalation airstream entering into the manifold 114 is preferentially directed towards the outlets of each atrium. Directing the separated inhalation airstream into the open blister pockets 164A, 164B in this way reduces turbulent energy in the airflow at this stage, and therefore reduces overall airflow resistance of the inhaler device 100. Overall airflow resistance allows patients to achieve higher flow rates for the same inhalation pressure, which may improve efficacy of drug delivery.

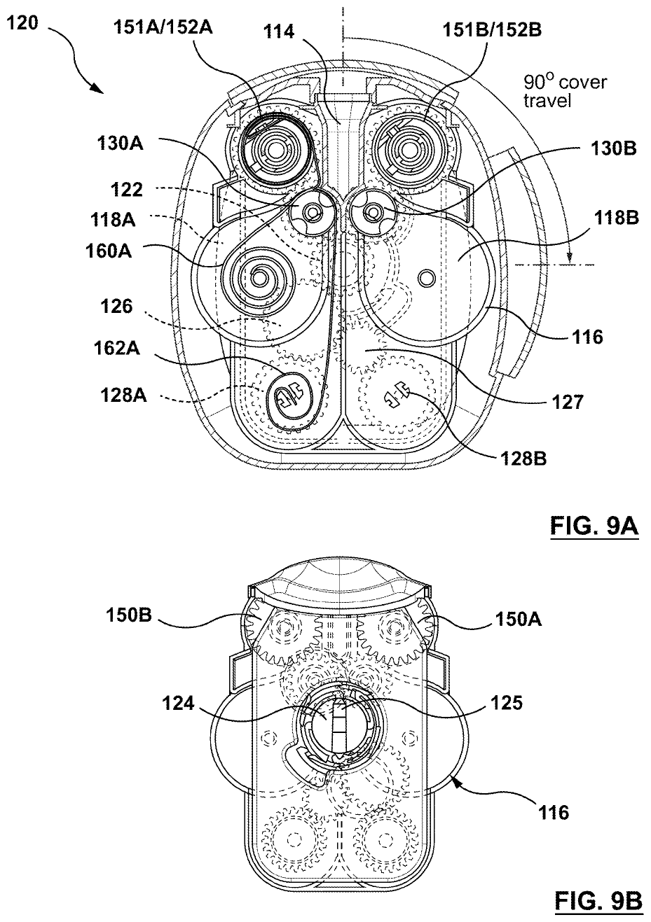

Turning now to FIGS. 9A and 9B, the dispensing subassembly 120 of the inhaler device 100 is described in more detail. The dispensing subassembly 120 is configured to advance each blister strip 160A, 160B and open a pocket 164A, 164B thereof each time the mouthpiece cover 108 is fully opened by the user. The first and second blister strips 160A, 160B are disposed within first and second compartments 118A, 118B within the housing 102. More particularly, compartments 118A, 118B are formed via an internal chassis 116 disposed within the housing 102. Via the dispensing subassembly 120, successive pockets 164A, 164B of each blister strip 160A, 160B are guided towards the manifold 114 which is disposed along a centerline, or disposed approximately along the centerline, of the inhaler device 100. When positioned at the manifold 114, a pocket 164A, 164B of each blister strip 160A, 160B has been opened and the powdered medicaments 168A, 168B within the opened pocket of each blister strip 160A, 160B is available for inhalation. The empty bottom sheets 162A, 162B and the top sheets 166A, 166B of the blister strips 160A, 160B are coiled up by the dispensing subassembly 120 as described herein. FIG. 9A is a front view of the inhaler device 100 with the mouthpiece cover 108 in the open position and a front half or portion of the housing 102 of the inhaler device 100 is removed for sake of illustration only. FIG. 9B is a rear view of the inhaler device 100 with the mouthpiece cover 108 and the housing 102 removed for sake of illustration only.

The dispensing subassembly 120 includes a central driver gear 122, a ratchet mechanism 124, a first idler or intermediate gear 126, a second idler or intermediate gear 127, first and second bottom sheet take-up gears 128A, 128B, first and second index gears 130A, 130B, and first and second top sheet take-up gears 150A, 150B. The first bottom sheet take-up gear 128A, the first index gear 130A, and the first top sheet take-up gear 150A are associated with advancement of the first blister strip 160A, while the second bottom sheet take-up gear 128B, the second index gear 130B, and the second top sheet take-up gear 150B are associated with advancement of the second blister strip 160B.

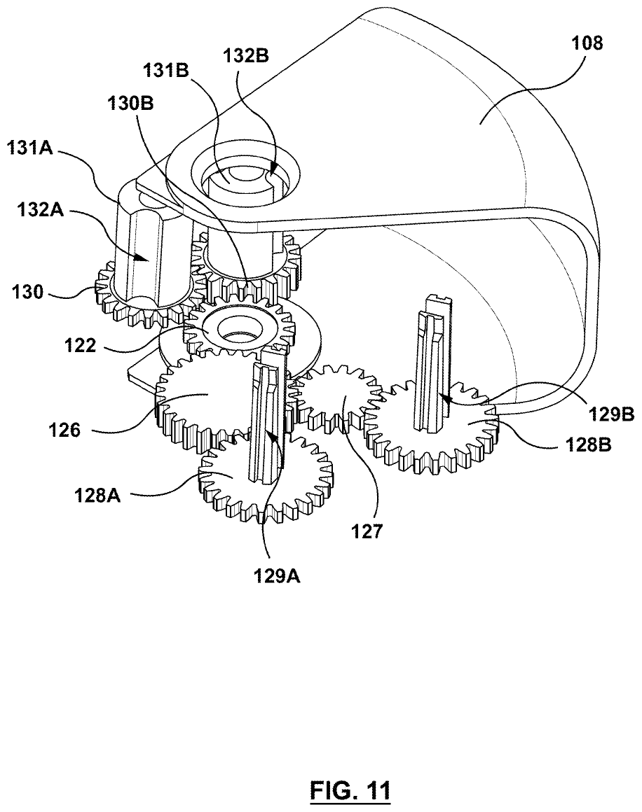

The first and second index gears 130A, 130B are attached to or integrally formed with first and second index spools 131A, 131B, respectively, as shown in FIG. 11. Each of first and second index spools 131A, 131B include a pair of recesses 132A, 132B, respectively, thereon. Each recess of the pair of recesses 132A, 132B is configured to receive a pocket 164A, 164B, respectively, of the blister strips 160A, 160B. Via rotation of the first and second index gears 130A, 130B, the first and second index spools 131A, 131B rotate and operate to move a recess 132A, 132B, respectively, adjacent to or in juxtaposition with the manifold 114. As the blister strips 160A, 160B are advanced by the index spools 131A, 131B, the top sheets 166A, 166B of the blister strips 160A, 160B are peeled away from the bottom sheets 162A, 162B of the blister strips such that a pocket 164A, 164B thereof adjacent to the manifold 114 is opened and the powdered medicaments 168A, 168B therein is available for entrainment. More particularly, in order to expose the powdered medicaments 168A, 168B for each dose, the top sheets 166A, 166B are peeled away from the bottom sheets 162A, 162B, respectively, such that a pocket 164A, 164B in each bottom sheet 162A, 162B is opened or uncovered. Uncovering or opening of a pocket 164A, 164B is achieved by relative rotation between the index spools 131A, 131B and the top sheet take-up gears 150A, 150B. The index spools 131A, 131B essentially grip the bottom sheets 162A, 162B, respectively, and the top sheet take-up gears 150A, 150B, respectively, essentially grip the top sheets 166A, 166B. When the index spools 131A, 131B and the top sheet take-up gears 150A, 150B rotate relative to each other, the bottom sheets 162A, 162B and the top sheets 166A, 166B are peeled apart from each other. As will be described in more detail herein, the index spools 131A, 131B and the top sheet take-up gears 150A, 150B are driven via gearing to rotate in opposite directions, such that each top sheet 166A, 166B is peeled away from its respective bottom sheet 162A, 162B as the gears are driven. The dispensing subassembly 120 thus causes the respective leading pocket 164A, 164B to be opened and positioned into fluid communication with manifold 114 so that the powdered medicaments 168A, 168B of the opened pockets 164A, 164B are available for inhalation.

The first and second bottom sheet take-up gears 128A, 128B operate to wind up the empty bottom sheets 162A, 162B, respectively, of the blister strips 160A, 160B. As shown in FIG. 11, the first and second bottom sheet take-up gears 128A, 128B are attached to or integrally formed with spindles 129A, 129B, respectively. Via rotation of the first and second bottom sheet take-up gears 128A, 128B, the first and second spindles 129A, 129B rotate and operate to take-up the bottom sheets 162A, 162B as the inhaler device 100 is operated. The end of each bottom sheet 162A, 162B is anchored to the first and second bottom sheet take-up gears 128A, 128A such that progressive rotation of the first and second bottom sheet take-up gears 128A, 128B results in the bottom sheets 162A, 162B being wound therearound into a tight coil.

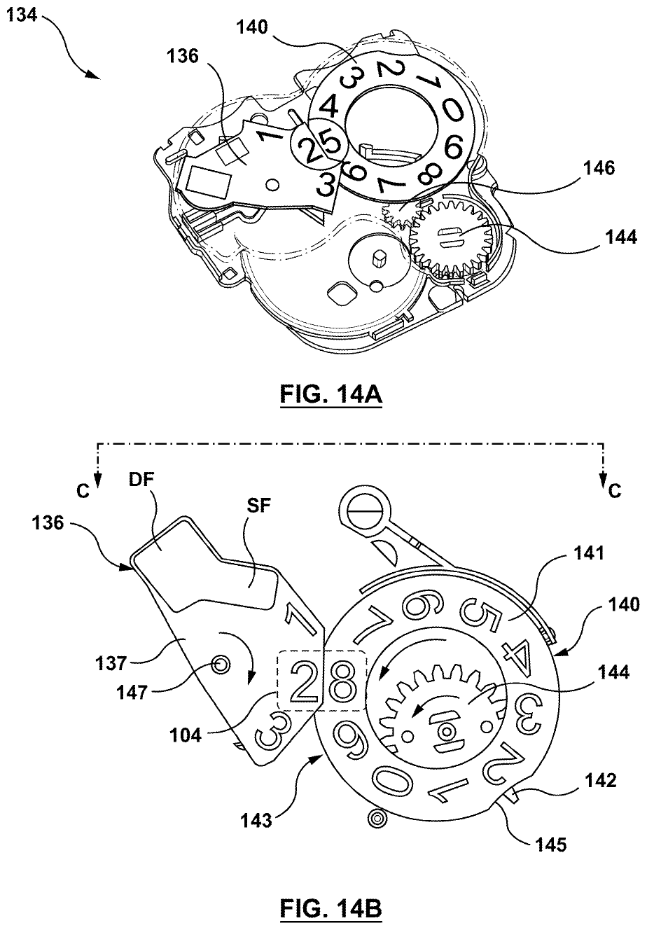

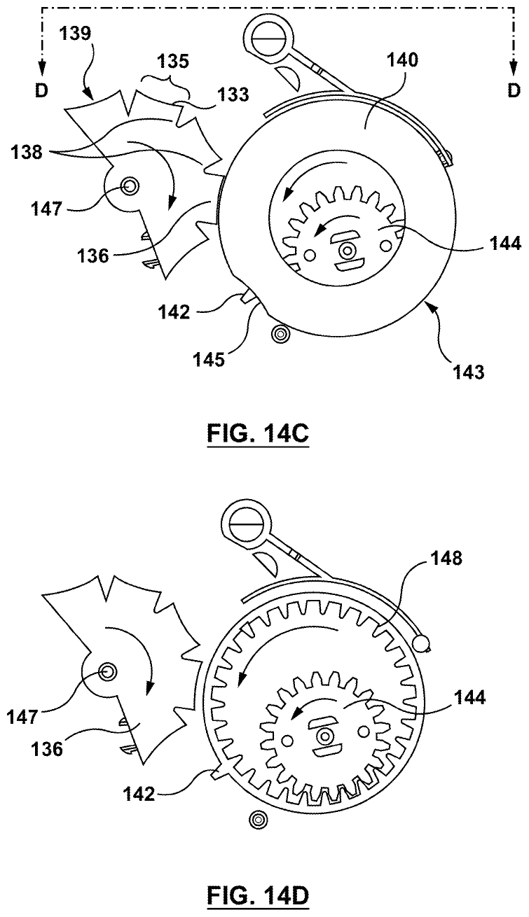

The first and second top sheet take-up gears 150A, 150B operate to wind up the top sheets 166A, 166B, respectively, of the blister strips 160A, 160B. As will be described in more detail herein with respect to FIGS. 14A-14E, the first and second top sheet take-up gears 150A, 150B are coupled to take-up hubs 152A, 152B, respectively. Via rotation of the first and second top sheet take-up gears 150A, 150B, the first and second take-up hubs 152A, 152B rotate and operate to take or wind up the top sheets 166A, 166B as the inhaler device 100 is operated. The end of each top sheet 166A, 166B is anchored to the first and second take-up hubs 152A, 152B such that progressive rotation of the first and second top sheet take-up gears 150A, 150B results in the top sheets 166A, 166B being wound therearound into a tight coil.

The central driver gear 122 of the dispensing subassembly 120 is attached to the mouthpiece cover 108 via the ratchet mechanism 124. The ratchet mechanism 124 is shown removed from the inhaler device 100 in FIG. 10. The ratchet mechanism 124 includes a ratchet 125 which is attached to the mouthpiece cover 108 and a ratchet gear 123 that is formed with or attached to the central driver gear 122. When the mouthpiece cover 108 is opened, the ratchet 125 is driven in the second opposing direction with the mouthpiece cover 108. The ratchet 125 in turn drives the ratchet gear 123 in the second opposing direction to advance or actuate the dispensing subassembly 120. When the mouthpiece cover 108 is returned to its closed position, the dispensing subassembly 120 is not advanced or actuated and remains stationary. An opening motion of the mouthpiece cover 108 is therefore transmitted to the central driver gear 122 but a closing motion of the mouthpiece cover 108 is not transmitted to the central driver gear 122.

More particularly, the ratchet gear 123 includes a plurality of circumferentially-spaced apart interior stop faces 123A and exterior stop faces 123B around an outer perimeter or edge thereof. The ratchet 125 includes a plurality of flexible ratchet arms 125A, which are configured to interact with the circumferentially-spaced apart interior stop faces 123A of the ratchet gear 123. The ratchet 125 rotates in a first direction with the mouthpiece cover 108 when the mouthpiece cover 108 is moved from a closed first position to an open second position. When rotating in the first direction, the ratchet arms 125A engage and drive the circumferentially-spaced apart interior stop faces 123A of the ratchet gear 123 as shown in FIG. 10B such that torque is transmitted to the central driver gear 122. Since the ratchet gear 123 is attached to or formed with the central driver gear 122, the central driver gear 122 rotates in the first direction concurrently with the mouthpiece cover 108. As described above, the movement of the mouthpiece cover 108 to the second position results in the opening and positioning of a pocket 164A, 164B of each blister strip 160A, 160B for subsequent simultaneous inhalation of the powdered medicaments 168A, 168B by the patient.

When the mouthpiece cover 108 is returned to its closed first position, however, the reverse rotation of the ratchet 125 is not transmitted to the central driver gear 122 because the ratchet arms 125A do not interact with the interior stop faces 123A of the ratchet gear 123. More particularly, when the ratchet 125 is rotated in the second opposing direction (i.e., counter clockwise), the ratchet arms 125A deflect radially inwards and no significant torque is transferred to the ratchet gear 123 and the central driver gear 122. Frictional drag between the ratchet 125 and ratchet gear 123 may tend to briefly drag the ratchet gear 123 in the second opposing direction (i.e., counter clockwise), but back-winding is prevented by interaction between one of the exterior stop faces 123B of the ratchet gear 123 and a flexible arm 121 in a retainer plate of the inhaler device 100, as shown in FIG. 10A.

In an embodiment, a detent (not shown) may be disposed between an inner surface of the mouthpiece cover 108 and an outer surface the housing 102. The detent may be a mating protrusion and groove that is configured to temporarily resist or prevent the movement of the mouthpiece cover 108 relative to the housing 102 until a user applies a force to the mouthpiece cover 108 (i.e., when opening the mouthpiece cover 108) to release the detent by causing one of the mating features of the detent to exit or move beyond the other of the mating features of the detent. For example, a protrusion, bump or other raised structure may be formed on the outer surface of the housing 102 and a mating groove, dimple or other indentation structure may be formed on the inner surface of the mouthpiece cover 108. Alternatively, the protrusion, bump or other raised structure may be formed on the inner surface of the mouthpiece cover 108 and a mating groove, dimple or other indentation structure may be formed on the outer surface of the housing 102. The groove is configured to receive the indentation when the mouthpiece cover 108 is in the closed position of FIG. 1A. When a user applies a force sufficient to overcome the friction between the mating protrusion and groove, the mouthpiece cover 108 begins to open and move away from the closed position of FIG. 1A. The detent is configured to prevent or deter unintentional openings of the mouthpiece cover 108. In addition, the detent is configured to account for rotational clearances or tolerances within the ratchet mechanism 124 such that the dispensing and counter mechanisms in the inhaler device 100 are actuated upon the first or initial movement of the mouthpiece cover 108 away from the closed position towards the open position.

As best shown on FIG. 11, the central driver gear 122 directly or indirectly drives the remaining gears of the dispensing subassembly 120. This gear train arrangement provides for incremental indexing or advancement of the blister strips 160A, 160B via the first and second index gears 130A, 130B and also provides for winding of the top and bottom sheets of the blister strips 160A, 160B (via the top sheet take-up gears 150A, 150B and the bottom sheet take-up gears 128A, 128B, respectively) due to rotational movement in the first direction of the mouthpiece cover 108 from its closed first position to its open second position. As the central driver gear 122 rotates in the first direction with the mouthpiece cover 108, the central driver gear 122 mates with or directly drives the second index gear 130B to rotate in a second opposing direction. The second spool 131B, and the second blister strip 160B advanced thereby, thus also rotates in the second opposing direction. As the second index gear 130B rotates in the second opposing direction, the second index gear 130B mates with or directly drives the first index gear 130A to rotate in the first direction. The first spool 131A, and the first blister strip 160A advanced thereby, thus also rotates in the first direction.

In an embodiment, a first direction is clockwise, and a second opposing direction is counter-clockwise. In the depicted embodiment, the central driver gear 122 rotates in a clockwise direction when the mouthpiece cover 108 is opened. As such, the second index gear 130B (along with the second spool 131B and the second blister strip 160B) rotate in a counter-clockwise direction and the first index gear 130A (along with the first spool 131A and the first blister strip 160A) rotate in a clockwise direction. As will be appreciated by one of ordinary skill in the art, however, the first direction may alternatively be counter-clockwise and the second opposing direction may be clockwise so long as the gear train formed by the dispensing subassembly 120 causes the first blister strip 160A to move or advance in an opposite direction from the second blister strip 160B. In addition, as will be appreciated by one of ordinary skill in the art, the gear train formed by the dispensing subassembly 120 may include one or more idler gears (not shown) which alter the sequence of rotation between the central driver gear 122, the second index gear 130B and the first index gear 130A. The presence of such idler gears do not impact the overall function of the gear train, so long as the gear train causes the first blister strip 160A to move or advance in an opposite direction from the second blister strip 160B. For example, an idler gear (not shown) may be disposed between the central driver gear 122 and the first index gear 130A and the central driver gear 122 (rotating in the first direction) directly drives the idler gear to rotate in the second opposing direction. The idler gear may be positioned to directly drive the first index gear 130A in the first direction, and the first index gear 130A directly drives the second index gear 130B in the second opposing direction.

In order to keep tension applied to the blister strips 160A, 160B, the first and second bottom sheet take-up gears 128A, 128B rotate concurrently with and in the same direction as the first and second index gears 130A, 130B, respectively, and the first and second top sheet take-up gears 150A, 150B rotate concurrently with and in the opposite direction as the first and second index gears 130A, 130B, respectively. As the central driver gear 122 rotates in the first direction, the central driver gear 122 mates with or directly drives the first idler gear 126 to rotate in the second opposing direction and the first idler gear 126 mates with or directly drives the first bottom sheet take-up gear 128A to rotate in the first direction. The first spindle 129A, and the bottom sheet 162A wound thereby, thus also rotate in the first direction to wind or take-up the empty bottom sheet 162A of the first blister strip 160A as the first blister strip 160A is advanced by the dispensing subassembly 120. In addition, the first top sheet take-up gear 150A interacts with or is driven by the first index gear 130A. Since the first index gear 130A rotates in the first direction, the first top sheet take-up gear 150A is driven to rotate in the second opposing direction to wind up the top sheet 166A of the first blister strip 160A.

As the central driver gear 122 rotates in the first direction, the first idler gear 126 is driven to rotate in the second opposing direction as described above and the first idler gear 126 mates with or directly drives the second idler gear 127 to rotate in the first direction. The second idler gear 127 mates with or directly drives the second bottom sheet take-up gear 128B to rotate in the second direction. The second spindles 129B, and the bottom sheet 162B wound thereby, thus also rotate in the second direction to wind or take-up the empty bottom sheet 162B of the second blister strip 160B as the second blister strip 160B is advanced by the dispensing subassembly 120. In addition, the second top sheet take-up gear 150B interacts with or is driven by the second index gear 130B. Since the second index gear 130B rotates in the second opposing direction, the second top sheet take-up gear 150B is driven to rotate in the first direction to wind up the top sheet 166B of the second blister strip 160B.

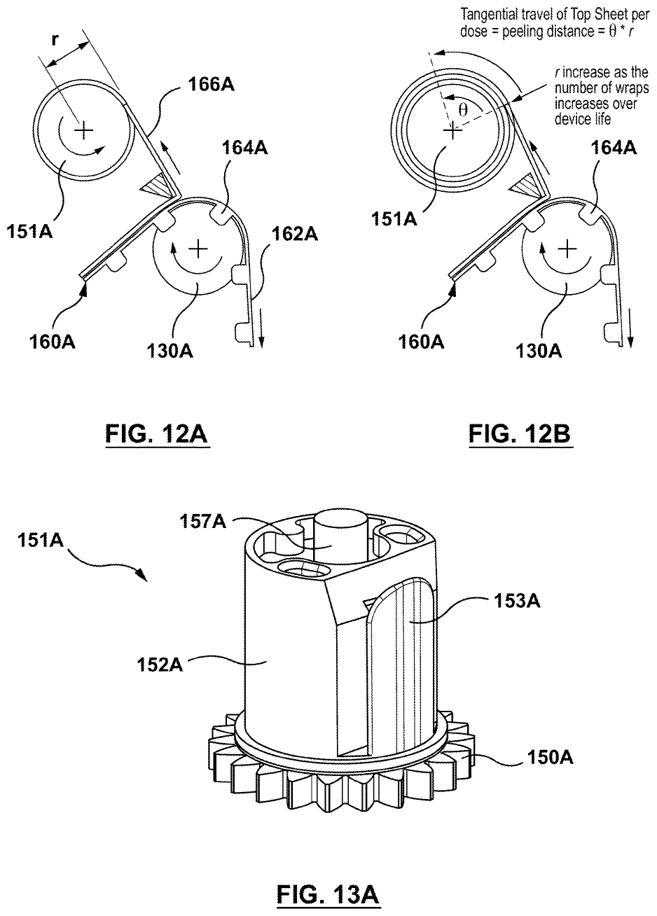

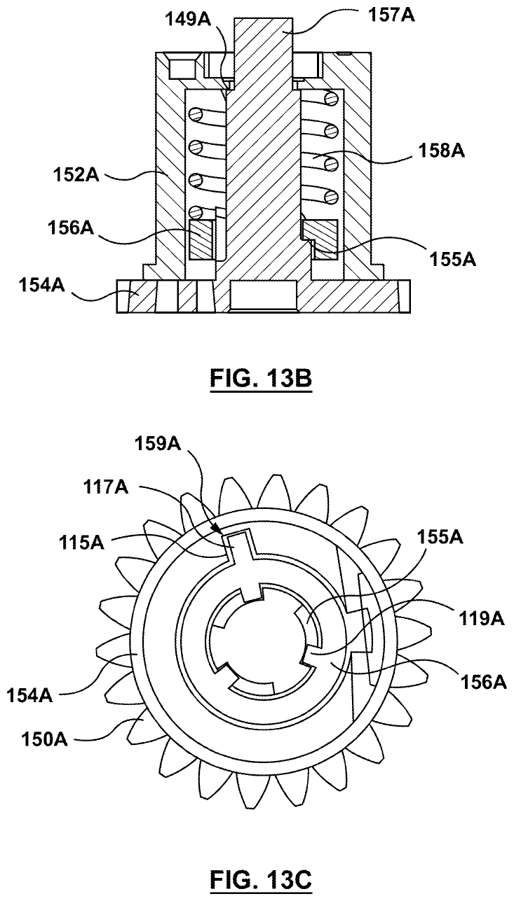

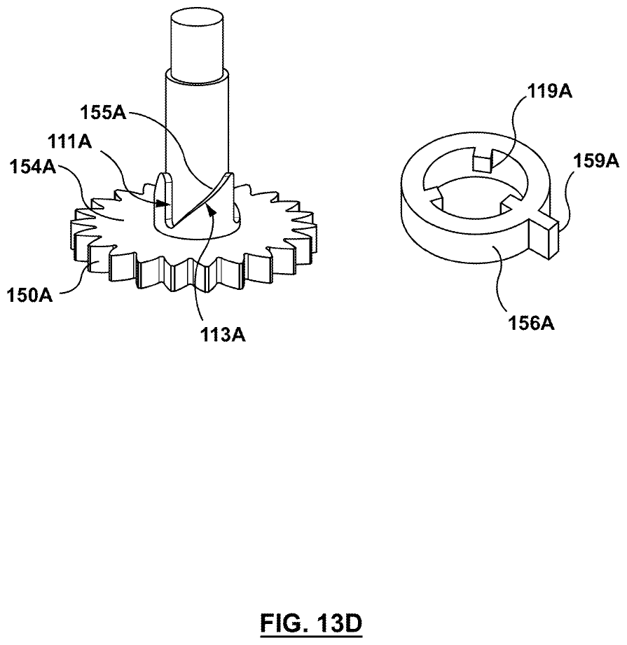

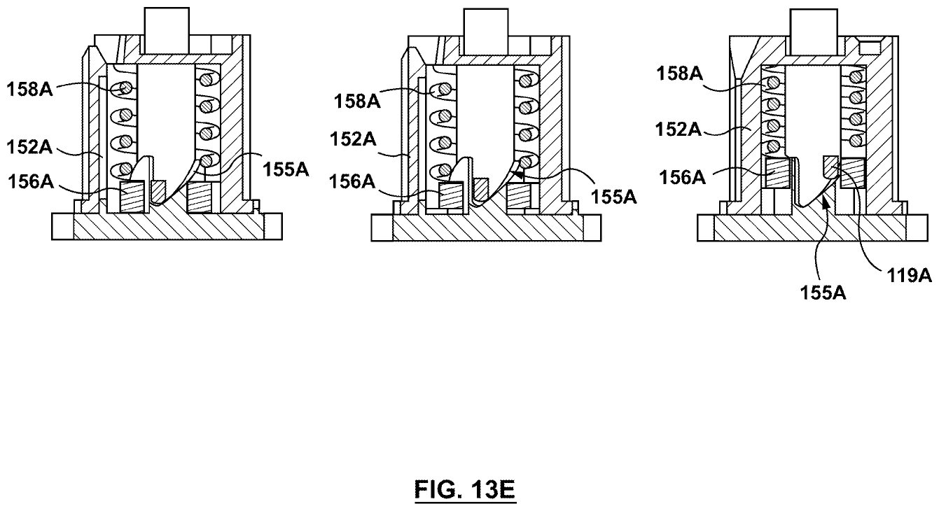

Turning now to FIGS. 12A and 12B and 13A-13E, first and second tensioning mechanisms 151A, 151B will be described in more detail. The tensioning mechanisms 151A, 151B function to peel the top sheets 166A, 166B from the first and second blister strips 160A, 160B, respectively, in a manner that maintains consistent peeling distance or amount over the device lifetime. More particularly, the tensioning mechanisms 151A, 151B ensure that the peeling distances of the top sheets 166A, 166B are configured to properly open the pockets 164A, 164B for each dose in order to achieve effective dispensing of the powdered medicaments 168A, 168B to the user. A pocket may not be fully exposed if the peeling distance or amount is too low, which makes it more difficult to achieve sufficient evacuation of the powdered medicament disposed therein upon inhalation. Further, if the peeling distance or amount is too high, the next or following pocket may be prematurely exposed and there is a risk of some medicament disposed therein being lost, which could result in an under dose on the next dispense. The tensioning mechanisms 151A, 151B also function to maintain sheet tension of the top sheets 166A, 166B over the device lifetime. The top sheets 166A, 166B need to be under consistent tension to ensure proper operation of the inhaler device 100. The tension of each top sheet 166A, 166B is linked to the force required by the user to operate the inhaler device 100 and move the mouthpiece cover 108. Ensuring consistent tension in the top sheets 166A, 166B thus provides a more consistent user experience over the device lifetime. Maintaining consistent tension in the top sheets 166A, 166B also results in more consistent and lower peak mechanical stress in the top sheets 166A, 166B and the surrounding components, which reduces the risk of mechanical failure during use.

The first tensioning mechanism 151A is associated with the first top sheet take-up gear 150A, for winding up the top sheet 166A of the first blister strip 160A, and the second tensioning mechanism 151B is associated with a second top sheet take-up gear 150B (see FIGS. 4A and 4B), for winding up the top sheet 166B of the second blister strip 160B. Only first tensioning mechanism 151A is described herein for sake of brevity, as the second tensioning mechanism 151B operates in the same manner. It will be apparent to those of ordinary skill in the art that certain features or components of the second tensioning mechanism 151B (i.e., the cam surface described herein) will be modified to extend in an opposing direction from the description below such that the second tensioning mechanism 151B is configured to operate in an opposite direction than the first tensioning mechanism 151A. Advantageously, the first tensioning mechanism 151A and the second tensioning mechanism 151B use some components of identical design (i.e., the compression spring and the take-up hub described herein), which may reduce the cost of manufacturing and assembly compared to other inhalation devices in which all components need to be manufactured individually for each side of the device in order to operate in opposite directions.

The tensioning mechanism 151A includes the first top sheet take-up gear 150A, the take-up hub 152A having a hook 153A integrally formed thereon or fixed thereto, a base 154A having a cam surface 155A integrally formed thereon or fixed thereto, a nut 156A, a shaft 157A, and a compression spring 158A that extends or is disposed between the nut 156A and a top end of the take-up hub 152A. The compression spring 158A biases the nut 156A downwards, towards the base 154A, into the cam surface 155A. The compression spring 158A is disposed about or around the shaft 157A and is longitudinally or axially adjacent to the nut 156A, and the take-up hub 152A is disposed about or around the nut 156A and the compression spring 158A. Stated another way, the take-up hub 152A encircles or surrounds the nut 156A and the compression spring 158A which are contained therein.

The top sheet 166A of the first blister strip 160A is secured or attached to the take-up hub 152A via the hook 153A such that as the take-up hub 152A rotates, the top sheet 166A of the first blister strip 160A wraps around it. The hook 153A is configured to attach to a leading end of the top sheet 166A such that rotation of the take-up hub 152A results in winding or wrapping of the top sheet 166A around the take-up hub 152A. Since the take-up hub 152A completes multiple rotations over the lifetime of the inhaler device 100, the top sheet 166A of the first blister strip 160A wraps over itself multiple times, causing its radial position on the take-up hub 152A to increase, i.e., a radial distance of each subsequent winding of the top sheet 166A from the take-up hub 152A increases with each winding. Since rotation of the base 154A corresponds to the rotation of the first top sheet take-up gear 150A, the base 154A rotates a fixed amount for each dose and the distance that the top sheet 166A of the first blister strip 160A is peeled relative to the bottom sheet 162A is determined by the tangential travel of the top sheet 166A of the first blister strip 160A at the take-up hub 152A. This tangential travel may be calculated as θ*r, where θ is the angle of rotation of the take-up hub 152A per dose, and r is the radial position of the top sheet 166A of the first blister strip 160A on the take-up hub 152A. As the radial position of the top sheet 166A of the first blister strip 160A increases, this tangential distance therefore increases for a fixed rotation of the take-up hub 152A and, without any modification of the mechanism, the top sheet 166A of the first blister strip 160A would be peeled further relative to the bottom sheet 162A later in the device life. This is known as the wrapping effect and is illustrated via a comparison of FIG. 12A and FIG. 12B. FIG. 12A is a schematic view of the first blister strip 160A early in the device life, and FIG. 12B is a schematic view of the first blister strip 160A late in the device life. FIG. 12B illustrates how the effective diameter of the take-up hub 152A increases as the first blister strip 160A is wrapped around the take-up hub 152A.

In order to ensure that the top sheet 166A of the first blister strip 160A is peeled the same amount each dose and compensate for the wrapping effect, the tensioning mechanism 151A includes the cam surface 155A, the nut 156A, and the compression spring 158A. The function of the cam surface 155A, the nut 156A, and the compression spring 158A is to provide a constant driving tension to the top sheet 166A over the entire strip length. As will be described in more detail herein, increased tension along the top sheet 166A results in rotation of the take-up hub 152A relative to the base 154A to reduce the tension along the top sheet 166A. When the take-up hub 152A rotates relative to the base, the compression spring 158A is compressed and axial compression of the compression spring 158A is transferred to torque applied to the take-up hub 152a.

With reference to FIGS. 13B-13D, the structure of the tensioning mechanism 151A will be described in more detail. The base 154A is attached to or formed integrally with the first top sheet take-up gear 150A to rotate as an assembly when the first top sheet take-up gear 150A is rotationally driven. For example, the base 154A may include a plurality of gear teeth integrally formed with or fixed to an outer circumferential surface thereof to form the first top sheet take-up gear 150A. The shaft 157A extends from the base 154A and is attached to or formed integrally with the base 154A to rotate therewith. Thus, the shaft 157A, the base 154A, and the first top sheet take-up gear 150A rotate as an assembly when the first top sheet take-up gear 150A is rotationally driven.

The nut 156A is disposed between and coupled to each of the take-up hub 152A and the base 154A. The nut 156A is disposed about or around the shaft 157A and is coupled to the base 154A via at least one inwardly-extending rib 119A that projects or extends radially inwards from an inner circumferential surface of the nut 156A. In an embodiment, the nut 156A includes a plurality of inwardly-extending ribs 119A that act as a cam follower. The plurality of inwardly-extending ribs 119A are preferably circumferentially spaced apart in equal increments. More particularly, as best shown on FIG. 13C, the inwardly-extending ribs 119A of the nut 156A are disposed onto and engage the cam surface 155A of the base 154A. Further, the nut 156A is coupled to the take-up hub 152A via a splined coupling 159A such that the take-up hub 152A rotates with the nut 156A and no relative rotation is permitted therebetween. Stated another way, due to the splined coupling 159A, the take-up hub 152A is rotationally locked to the nut 156A such that the nut 156A and the take-up hub 152A rotate as an assembly. As best shown on FIG. 13C, the splined coupling 159A includes an outwardly-extending rib 117A which projects or extends radially outwards from an outer circumferential surface of the nut 156A and is received within an axial slot 115A of the take-up hub 152A. The outwardly-extending rib 117A is permitted to slide or move in an axial direction along the axial slot 115A such that the nut 156A is permitted to slide or move in an axial direction relative to the take-up hub 152A, but the outwardly-extending rib 117A does not permit the nut 156A to rotate relative to the hub 152A.

When the base 154A rotates in the second opposing direction with the first top sheet take-up gear 150A, the nut 156A and the take-up hub 152A also rotate in the second opposing direction due to the interaction between the nut 156A with the compression spring 158A and the cam surface 155A of the base 154A. More particularly, as the base 154A is being rotationally driven in the second opposing direction, the take-up hub 152A is configured to rotate in the second opposing direction via engagement of the inwardly-extending ribs 119A of the nut 156A with the cam surface 155A. As shown on FIG. 14D, the cam surface 155A includes alternating sections of vertical surfaces 111A and angled or inclined surfaces 113A. Vertical surfaces 111A extend generally parallel to a longitudinal axis of the shaft 157A. As the base 154A is being rotationally driven in the second opposing direction (i.e., the counter-clockwise direction in this embodiment), the nut 156A and the take-up hub 152A rotationally locked therewith rotate with the base 154A in the second opposing direction. Interaction between the compression spring 158A, the nut 156A and the cam surface 155A result in a torque being applied from the cam surface 155A to the nut 156A, in the direction that would push the nut 156A down the cam surface 155A. The compression spring 158A is pushing the nut 156A against the inclined surfaces 113A of the cam surface 155A, which results in torque acting on the take-up hub 152A that drives the take-up hub 152A in the second opposing direction. As a result of this interaction when the base 154A moves counter-clockwise, the nut 156A and the take-up hub 152A also rotate counter-clockwise with the base 154A. It will be understood by one of ordinary skill in the art that the pattern of the cam surface 155A is exemplary. A thread or other ramped surface may be utilized as the cam surface 155A.

Although the take-up hub 152A rotates in the second opposing direction with the base 154A due to the nut 156A interacting with the compression spring 158A and the cam surface 155A of the base 154A, relative rotation between the take-up hub 152A and the base 154A is permitted. More particularly, when sufficient torque is applied between the take-up hub 152A and the base 154A, the take-up hub 152A will rotate relative to the base 154A. Since the take-up hub 152A is permitted to rotate relative to the base 154A in a direction that will reduce tension in the top sheet 166A (i.e., in the first direction), this relative rotation stabilizes or balances the tension in the top sheet 166A via deflection of the compression spring 158A. The tensioning mechanism 151A is therefore acting as a torsion or torque limiter between the take-up hub 152A and the base 154A to control the tension in the top sheet 166A of the first blister strip 160A.

In addition to the torque being applied from the cam surface 155A to the nut 156A, there is also a torque in the opposing direction acting on the nut 156A from its interaction with the take-up hub 152A via the outwardly-extending rib 117A. This opposing torque comes from the tension in the top sheet 166A, which is acting to apply torque to the take-up hub 152A. The two opposing torques on the nut 156A are in equilibrium, and as such the compression spring 158A (via the nut 156A and cam surface 155A) effectively balances the tension in the top sheet 166A. If the tension in the top sheet 166A increases, then the nut 156A will move further up the cam surface 155A and the spring force of the compression spring 158A will increase to compensate for the increased tension.

More particularly, due to the wrapping effect described above, as the take-up hub 152A rotates, the radial position of the top sheet 166A of the first blister strip 160A increases. As a result of this increased radial position, the take-up hub 152A attempts to peel a longer length of top sheet 166A and the tension on the top sheet 166A increases due to a change in the peeling angle between the top sheet 166A of the first blister strip 160A and the bottom sheet 162A of the first blister strip 160A. When tension on the top sheet 166A increases, the torque that the top sheet 166A applies to the take-up hub 152A increases as well. Rather than the torque continuing to increase, the take-up hub 152A will start to rotate relative to the base 154A in the second direction, and the nut 156A will move helically up the cam surface 155A of the base 154A, thereby compressing the compression spring 158A. More particularly, when the take-up hub 152A and the nut 156A rotationally locked thereto begin to rotate in the second direction due to the increased tension on the top sheet 166A, the base 154A and the cam surface 155A remain stationary and the inwardly-extending ribs 119A of the nut 156A move along the inclined surfaces 113A of the cam surface 155A in a direction towards the compression spring 158A, i.e., higher up the cam surface 155A. The compression spring 158A compresses as the nut 156A presses against it. As the nut 156A moves relative to the base 154A, the nut 156A also moves axially relative to the take-up hub 152A because the outwardly-extending rib 117A of the nut 156A is permitted to move axially within the axial slot 115A of the take-up hub 152A. Thus, rotation of the take-up hub 152A relative to the base 154A results in axial movement of the nut 156A relative to the take-up hub 152A and relative to the base 154A, and further axial movement of the nut 156A towards the compression spring 158A axially compresses the compression spring 158A. Since the take-up hub 152A is rotationally locked to the nut 156A via the splined coupling 159A, the nut 156A transfers the axial force of the compressed spring 158A into torque on the take-up hub 152A. The combination of the compression spring 158A, the nut 156A, and the cam surface 155A is thereby providing torque to react or counteract the relative rotation between the take-up hub 152A and the base 154A, which is acting to maintain consistent tension in the top sheet 160A.

The take-up hub 152A is axially constrained relative to the base 154A via a clip or retention feature 149A disposed between the shaft 157A and the take-up hub 152A. More particularly, since the compression spring 158A is acting to axially separate the take-up hub 152A and the base 154A, the retention feature 149A (best shown on FIG. 13B) is disposed between the top end of the take-up hub 152A and the compression spring 158A to maintain the correct relative axial position between the take-up hub 152A and the base 154A. The retention feature 149A may be a clip, a bayonet, or other component suitable to maintain the correct relative axial position between the take-up hub 152A and the base 154A. In another embodiment (now shown), the retention feature may be attached to the interior of the housing 102 to maintain the correct relative axial position between the take-up hub 152A and the base 154A.

FIG. 13E illustrates how the position of the nut 156A changes over the lifetime of the device. In its initial assembled state shown in the left image, prior to attachment of the take-up hub 152A to the first blister strip 160A, the nut 156A rests at the bottom end of the cam surface 155A. The cam surface 155A is designed such that, in this position, there is no resultant torque between the take-up hub 152A and the base 154A, even though there may be axial force from the compression spring 158A. When the device is assembled, the top sheet 166A is assembled under some tension to ensure that peeling is effective from the first dose and as a result, the nut 156A is lifted up the cam surface 155A slightly away from the vertical surfaces 111A of the cam surface 155A. More particularly, as shown in the middle image, when the device is assembled the top sheet 166A is attached to the take-up hub 152A, the take-up hub 152A is rotated relative to the base 154A, and the nut 156A moves up the cam surface 155A. The compression spring 158A is deflected or slightly compressed from its uncompressed length so that the compression spring 158A has a preload force. The preload force ensures that the tension in the top sheet 166A of the first blister strip 160A is sufficiently high to peel it away from the bottom sheet 162A of the first blister strip 160A at the start of the device life. The specifications of the compression spring 158A and the angle of the cam surface 155A should be configured to provide a minimum tension in the top sheet 166A of the first blister strip 160A that is higher than the maximum force required to peel the top sheet 166A of the first blister strip 160A from the bottom sheet 162A of the first blister strip 160A. In this assembled state, the combination of the compression spring 158A, the nut 156A, and the cam surface 155A provide a torque between the base 154A and the take-up hub 152A, which is reacted by tension in the top sheet 166A. Over the lifetime of the inhaler device 100 as shown in the right image, the tension in the top sheet 166A of the first blister strip 160A increases. Due to the increased tension of the top sheet 166A, the nut 156A moves further up the cam surface 155A so that the increased tension of the top sheet 166A is balanced by further deflection of the compression spring 158A.

The angle or slope of each inclined surface 113A of the cam surface 155A is configured to maintain consistent sheet tension of the top sheets 166A, 166B over the device lifetime. As described above, the top sheets 166A, 166B need to be under consistent tension to ensure proper operation of the inhaler device 100. In general, the angle or slope of each inclined surface 113A of the cam surface 155A is selected to ensure that the nut 156A moves along the cam surface 155A of the base 154A during operation of the inhaler device 100 and does not move beyond or past the vertical surfaces 111A. In an embodiment, the inclined surfaces 113A of the cam surface 155A extend at an angle between 35 degrees and 55 degrees relative to the longitudinal axis of the base 154A. In an embodiment, the inclined surfaces 113A of the cam surface 155A extend at an angle between 40 degrees and 50 degrees relative to the longitudinal axis of the base 154A. In an embodiment, the inclined surfaces 113A of the cam surface 155A extend at an angle of approximately 45 degrees relative to the longitudinal axis of the base 154A, with approximately as used herein including a tolerance of three degrees. In an embodiment, the inclined surfaces 113A of the cam surface 155A have a slope between 0.70 and 1.0. In an embodiment, the inclined surfaces 113A of the cam surface 155A have a slope between 0.80 and 0.95. In another embodiment, the inclined surfaces 113A of the cam surface 155A have a slope between 0.7 and 1.4. In another embodiment, the inclined surfaces 113A of the cam surface 155A have a slope between 1.0 and 1.4. The slope of the inclined surfaces 113A of the cam surface 155A may be constant over the length of the inclined surface, or may vary over the length of the inclined surface.

The radial width of the inclined surface 113A of the cam surface 155A is configured to optimize the amount of friction between the nut 156A and the cam surface 155A. In general, larger dimensions of the radial width of the inclined surface 113A of the cam surface 155A result in increased friction between the components while smaller dimensions of the radial width of the inclined surface 113A of the cam surface 155A may result in the nut 156A undesirably falling off the cam surface 155A. In an embodiment, each inclined surface 113A of the cam surface 155A has a radial width between 1 mm and 3 mm. In an embodiment, each inclined surface 113A of the cam surface 155A has a radial width between 1.5 mm and 2.5 mm. In an embodiment, each inclined surface 113A of the cam surface 155A has a radial width of approximately 2 mm, with approximately as used herein including a tolerance of 0.2 mm.

Turning now to FIGS. 14A-14D, the counter subassembly 134 will be described in more detail. The dispensing subassembly 120 advantageously directly drives the counter subassembly 134, so that the dose counter is incremented automatically and simultaneously with a medicament dose being indexed or delivered by the inhaler device 100. As such, beyond operating the mouthpiece cover 108 to actuate the dispensing subassembly 120, the user is not required to perform any additional operating steps to update the dose counter. The dose counter is incremented automatically when the mouthpiece cover 108 is opened, so it is intuitive to the user what the dose counter relates to.

The counter subassembly 134 includes a first count wheel or units ring 140 and a second count component or tens mechanism 136. The first count wheel 140, driven from the dispensing subassembly 120 of the inhaler device 100 to rotate a fixed angle per dose, and in this embodiment is configured to display a second digit of a two-digit number of the available dose count. The second count component 136 is driven intermittently from the first count wheel 140 such that it rotates a fixed angle per revolution of the first count wheel 140, as will be described in more detail below. In the present embodiment, the second count component 136 displays a first digit of the two-digit number of the available dose count. As such, the first count wheel 140 and the second count component 136 collectively display a number of doses remaining within the inhaler device 100, as shown in FIGS. 14A and 14B. Although the embodiments of the counter subassemblies described herein display a two-digit number, it will be understood by one of ordinary skill in the art that the counting indicia may be revised to display a three-digit number if the total number of doses in the inhaler device exceed one-hundred.

A front or indicia-displaying face or surface 141 of the first count wheel 140 includes counting indicia disposed thereon that includes units or ‘ones’ digits. More particularly, as shown in FIGS. 14A and 14B, the counting indicia of the first count wheel 140 includes the digits 0, 1, 2, 3, 4, 5, 6, 7, 8, 9 in a circular pattern near an outer peripheral edge of the indicia-displaying surface 141. The angle between each digit is the same as the angle of rotation of the first count wheel 140 per dose. The indicia-displaying surface 141 is planar.

A front or indicia-displaying face or surface 137 of the second count component 136 includes counting indicia disposed thereon that includes ‘tens’ digits. The counting indicia of the second count component 136 includes the digits 3, 2, 1 and may also include a single flag SF and a double flag DF near an outer peripheral edge of the indicia-displaying surface 137. The angle between each counting indicia or digit is the angle of rotation of the second count component 136 for every revolution of the first count wheel 140. The indicia-displaying surface 137 is planar.

The display window 104 in the housing 102 is positioned such that one digit on the first count wheel 140 and one digit on the second count component 136 are visible within the display window, and adjacent to each other to form a two-digit number. The displayed digit on the second count component 136 reflects or tracks the number of ‘tens’ of doses remaining in the inhaler device 100, while the displayed digit on the first count wheel 140 reflects or tracks the number of ‘ones’ of doses remaining. When the number of remaining doses is below ten, the second count component 136 will display the single flag SF in the display window 104 instead of or in place of a zero digit. In an embodiment, for example, the single flag SF may be a colored block with no digits thereon to indicate to the user that they are nearing the end of available doses within the inhaler device 100. When the number of remaining doses reaches zero, the second count component 136 will display the double flag DF in the display window 104 instead of a digit. In an embodiment, for example, the double flag DF may be a colored block with no digits thereon that covers the digit of the first count wheel 140 to give clear visual feedback to the user that no doses remain within the inhaler device 100. The double flag DF is configured to cover the digit of the first count wheel 140 when no doses remain within the inhaler device 100.

In the embodiment of FIGS. 14A-14D, the units or ‘ones’ digits are circumferentially disposed on the first count wheel 140 in a descending order in a first direction, and the ‘tens’ digits are disposed near a perimeter of the second count component 136 in a descending order in a second opposing direction. While the counter subassemblies described herein display a number of doses remaining within the inhaler device 100, it will be understood by one of ordinary skill in the art that the counter subassemblies may be modified to display a number of doses delivered by the inhaler device by reversing the order of the counting indicia disposed on the counting subassembly.

Turning now to FIGS. 14C and 14D, the structure and operation of the first count wheel 140 and the second count component 136 will be described in more detail. FIG. 14C is a sectional view taken along line C-C of FIG. 14B, which is along an intermediate position between the indicia-displaying surfaces and the opposing back surfaces of the count subassembly. FIG. 14D is a sectional view taken along line D-D of FIG. 14C, which is adjacent to the opposing back surfaces of the count subassembly. The relative terms “front” and “back” are utilized herein for sake of illustration only and relate to how an inhaler device is customarily positioned by a user during use with a front of the inhaler device including the display window for the counter mechanism.

The first count wheel 140 is an annular or ring-shaped component having the front or indicia-displaying surface 141 and an opposing back surface which includes a counter gear 148, as best shown in the sectional view of FIG. 14D. The counter gear 148 may be integrally formed on or attached to the first count wheel 140. An outer circumferential surface or side 143 extends between the front and back surfaces of the first count wheel 140. The outer circumferential side 143 may be stepped, with a smaller diameter along the back surface of the first count wheel 140 and a larger diameter along the front surface of the first count wheel 140. The stepped nature of the outer circumferential side 143 is apparent via a comparison of the relative diameters thereof in FIGS. 14C and 14D. Stated another way, the indicia-displaying surface 141 of the first count wheel 140 has a greater outer diameter than the counter gear 148.

Adjacent to the counter gear 148, the first count wheel 140 includes a single tooth or protrusion 142 extending radially outward from the outer circumferential side 143. Along the front or indicia-displaying surface 141 of the first count wheel 140, the outer circumferential side 143 is generally circular with a clearance cutout or indent 145 formed around the single tooth 142. The function of the clearance indent 145 will be described in more detail below. In another embodiment hereof (not shown), the first count wheel 140 may include more than a single tooth or protrusion 142. Thus, while at least one single tooth or protrusion is required and a single tooth is deemed to be sufficient and advantageously simplifies the design and reduces material costs of the first count wheel 140, embodiments hereof may include more than one tooth or protrusion.

The second count component 136 is configured to rotate around a pivot point 147. The second count component 136 is a non-annular or partial-disk component having the front or indicia-displaying surface 137 and an opposing back surface which includes a plurality of notches 138, as shown in the sectional views of FIGS. 14C and 14D. An outer surface or side 139 extends between the front and back surfaces of the second count component 136. The outer side 139 may be stepped, with a smaller radial dimension along the back surface of the second count component 136 and a larger radial dimension along the front surface of the second count component. The stepped nature of the outer side 139 is apparent via a comparison of the relative diameters thereof in FIGS. 14B and 14C.

The plurality of notches 138 are formed in the outer side 139 of the second count component 136 and do not extend through the front or indicia-displaying surface 137. In an embodiment, the second count component 136 includes four notches 138 but the number of notches is exemplary and depends on the capacity or total number of available doses within the inhaler device 100. Each notch 138 extends radially inward toward the pivot point 147 of the second count component 136 and is configured to mate with or receive the single tooth 142 of the first count wheel 140. The outer side 139 of the second count component 136 may be considered to include a plurality of segments 135, with each segment 135 extending between two neighboring or adjacent notches 138. Along each segment 135, the outer side 139 of the second count component 136 is concave and forms an inverted curved or arc-shaped indent 133 that is coincident with the outer circumferential side 143 of the first count wheel 140.

The first count wheel 140 is driven by the second bottom sheet take-up gear 128B of the dispensing subassembly 120 such that the first count wheel 140 rotates a fixed angle every time a dose is dispensed. More specifically, a transfer gear 144 is attached to an opposing end of the second spindle 129B of the second bottom sheet take-up gear 128B so that the transfer gear 144, the second spindle 129B, and the second bottom sheet take-up gear 128B rotate simultaneously as an assembly. When the mouthpiece cover 108 is opened, the transfer gear 144 thus rotates in the second opposing direction with the second bottom sheet take-up gear 128B. The transfer gear 144 mates with or directly drives an idler gear 146 to rotate in the first direction, and the idler gear 146 mates with or directly drives the counter gear 148 to rotate in the second opposing direction. Thus, the counter gear 148 rotates in the same direction as the transfer gear 144. As will be apparent to those of ordinary skill in the art, rotation of the transfer gear 144 and the counter gear 148 in the same direction may also be accomplished via a gear train in which the transfer gear 144 directly drives the counter gear 148 in the same direction. For example, as depicted in FIGS. 14B-14D, the desired rotation scheme is accomplished when the counter gear 148 is an internal gear and the transfer gear 144 is a spur or planetary gear so that the transfer gear 144 meshes with or directly drives the internal counter gear 148 in the same direction. The first count wheel 140 is attached to the counter gear 148 to rotate therewith as an assembly, and thus the first count wheel 140 rotates in the second opposing direction when the mouthpiece cover 108 is opened. Thus, various suitable gear train may be utilized herein such that the transfer gear 144 and the counter gear 148 rotate in the same direction.

As best shown on FIG. 14C, the second count component 136 is positioned adjacent or next to the outer circumferential side 143 of the first count wheel 140, such that the single tooth 142 of the first count wheel 140 engages a notch 138 of the plurality of notches 138 of the second count component 136 once per revolution of the first count wheel 140 to intermittently rotate the second count component 136. Stated another way, each time that the first count wheel 140 makes a complete revolution, the single tooth 142 engages a notch 138 of the second count component 136 and turns or rotates the second count component 136 a fixed amount. Thus, there is a fixed rotation of the second count component 136 once per revolution of the first count wheel 140. Since the first count wheel 140 directly drives the second count component 136, the second count component 136 rotates in an opposing direction than the first count wheel 140. In the embodiment of FIGS. 14A-14D, the second count component 136 is configured to rotate in the first direction and the first count wheel 140 is configured to rotate in the second opposing direction. The second count component 136 is stationary and does not rotate when the single tooth 142 of the first count wheel 140 is not engaged a notch 138 of the second count component 136.

The second count component 136 is disposed next to or side-by-side in a common plane with the first count wheel 140, and thus the second count component 136 rotates on a different axis of rotation than the first count wheel 140. Stated another way, the first count wheel 140 rotates about a first axis and the second count component 136 rotates about a second axis, with the second axis being parallel to and spaced apart from the first axis.

As stated above, along each segment 135, the outer side 139 of the second count component 136 is concave and forms the arc-shaped indent 133 that is coincident with the outer circumferential side 143 of the first count wheel 140. As best shown on FIG. 14C, the geometry of the arc-shaped indents 133 is coincident with the circular profile of the first count wheel 140 to prevent the second count component 136 from inadvertently rotating when not engaged with the first count wheel 140. Particular, this geometry or profile of the outer side 139 of the second count component 136 ensures that the second count component 136 does not rotate, and remains stationary, when the single tooth 142 of the first count wheel 140 is not engaged with one of the notches 138 of the second count component 136. Conversely, when the single tooth 142 of the first count wheel 140 is engaged with or received within one of the notches 138 of the second count component 136, the clearance indent 145 of the first count wheel 140 allows the second count component 136 to briefly rotate with the first count wheel 140 to change the tens display.

While having a relatively large and clear display of the number of available doses remaining within an inhaler device is an important consideration for readability, there is often a trade-off between the size of the dose counter display and the space that the dose counter mechanism takes up within the housing of the inhaler device. The counter subassembly 134 advantageously splits the two-digit number of available doses between multiple components, which makes it easier to achieve a larger number size in the same amount of space within the housing of the inhaler device. In addition, the counter subassembly 134 does not require any type of detent feature to hold or maintain the position thereof. For example, other dose counter mechanisms utilize a flexible arm as a detent feature to hold the position of the dose count mechanism, and some force is required to deflect the flexible arm in order to allow the dose count mechanism to increment. Such detent features cause an increase in the operating force of the device, which is undesirable. The counter subassembly 134 also does not require any type of intermediate components between the count components. By driving the second count component 136 directly from the first count wheel 140, the counter subassembly 134 is simplified and overall number of components of the inhaler device 100 is minimized to reduce the overall cost and complexity of the inhaler device 100. Further, elimination of an intermediate component between the count components means that there is a shorter tolerance chain for alignment between the first count wheel 140 and the second count component 136 which results in more consistent alignment between the first and second digits of the two-digit number of the display.

In the embodiment of FIGS. 14A-14D, as best shown on FIG. 14B, the front or indicia-displaying surface 137 of the second count component 136 overlaps or overlays a portion of the front or indicia-displaying surface 141 of the first count wheel 140. Thus, the ‘ones’ digits on the first count wheel 140 and the ‘tens’ digits on the second count component 136 are not coplanar. Stated another way, the ‘ones’ digits on the first count wheel 140 and the ‘tens’ digits on the second count component 136 are on different planes or levels, with the ‘tens’ digits being raised relative to the ‘ones’ digits, or to be disposed closer to the display window 104 of the inhaler device 100.

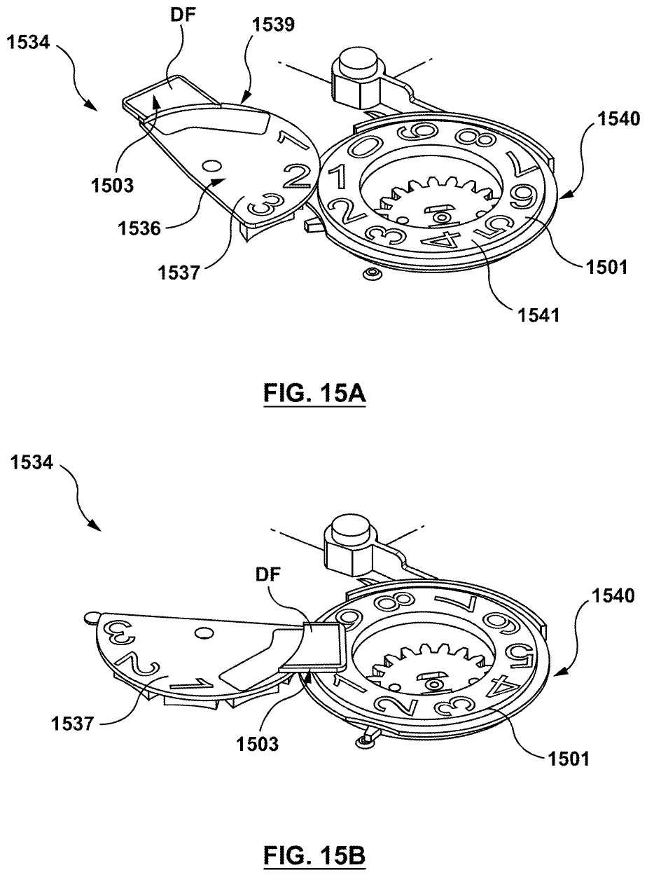

FIGS. 15A and 15B illustrate another embodiment of a counter subassembly 1534, in which the ‘tens’ digits are coplanar or level with the ‘ones’ digits. The counter subassembly 1534 is the same as the counter subassembly 134 described above, except for the differences described herein. One benefit of the configuration of the counter subassembly 1534 is having the ‘tens’ digits at the same level with the ‘ones’ digits may improve readability of the display, particularly at shallower viewing angles. A circular boss 1501 is integrally formed with or secured to a first count wheel 1540 to lift or raise up the ‘ones’ digits thereon to the same level as the ‘tens’ digits on a second count component 1536. The circular boss 1501 forms a front or indicia-displaying face or surface 1541 of the first count wheel 1540. In addition, a double flag DF on a second count component 1536 is disposed on a raised or stepped-up level 1503 that is integrally formed with or secured to the second count component 1536 so that the double flag DF is configured to cover a digit on the first count wheel 1540 at the end of device life as shown on FIG. 15B. More particularly, the raised or stepped-up level 1503 radially extends outward from an outer surface or side 1539 of the second count component 1536, so that the raised or stepped-up level 1503 extends over or overlaps a digit on the first count wheel 1540 at the end of device life. In this embodiment, a front or indicia-displaying face or surface 1537 of the second count component 1536 is coplanar with the front or indicia-displaying surface 1541 of the first count wheel 1540, except as noted for the raised or stepped-up level 1503.

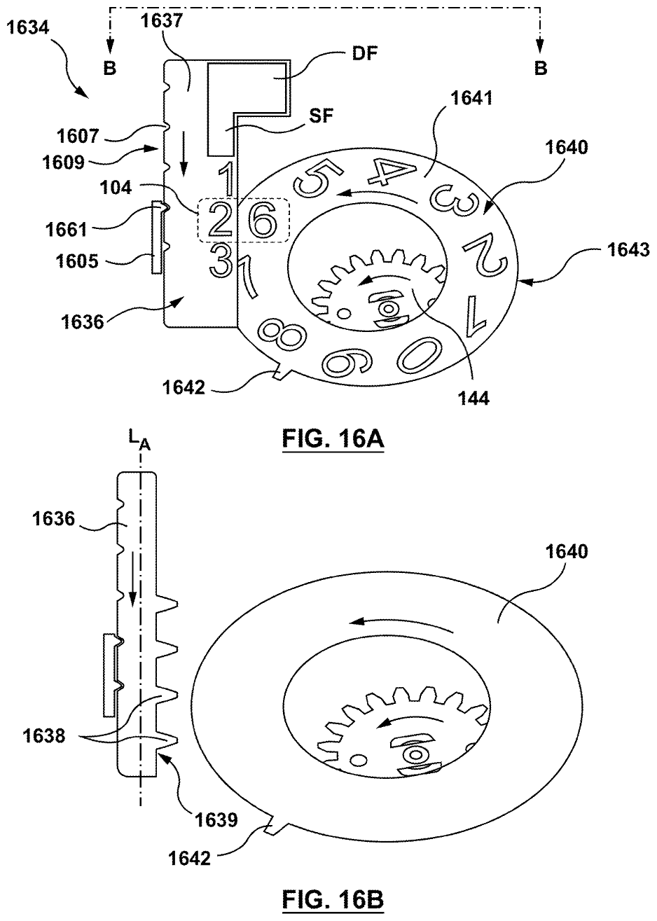

Another embodiment of a counter subassembly 1634 that may be utilized within the inhaler device 100 is depicted in FIGS. 16A and 16B. The counter subassembly 1634 includes a first count wheel or units ring 1640 and a second count component or tens mechanism 1636. The counter subassembly 1634 is similar to the counter subassembly 134 but a slider component which moves along a linear path replaces the rotatable second count component 136.

Similar to the counter subassembly 134, the dispensing subassembly 120 advantageously directly drives the counter subassembly 1634 so that the dose counter is incremented automatically and simultaneously with a medicament dose being indexed or delivered by the inhaler device 100. The first count wheel 1640, driven from the dispensing subassembly 120 of the inhaler device 100 to rotate a fixed angle per dose, is configured to display a second digit of a two-digit number of the available dose count. The first count wheel 1640 is the same as the first count wheel 140, except that the first count wheel 1640 does not include the clearance indent 145. The first count wheel 1640 includes a single tooth or protrusion 1642 extending radially outward from an outer circumferential side or surface 1643 thereof. In another embodiment hereof (not shown), the first count wheel 1640 may include more than a single tooth or protrusion 1642. Thus, while at least one single tooth or protrusion is required and a single tooth is deemed to be sufficient and advantageously simplifies the design and reduces material costs of the first count wheel 1640, embodiments hereof may include more than one tooth or protrusion.

The second count component 1636 is a slider element which is moved intermittently by the first count wheel 1640 such that it moves or translates a fixed amount per revolution of the first count wheel 1640, as will be described in more detail below. The second count component 1636 displays a first digit of a two-digit number of the available dose count. As such, the first count wheel 1640 and the second count component 1636 collectively display a number of doses remaining within the inhaler device 100, as shown in FIG. 16A. A front or indicia-displaying face or surface 1637 of the second count component 1636 includes counting indicia disposed thereon that includes ‘tens’ digits. The counting indicia of the second count component 1636 includes the digits 3, 2, 1 and may also include a single flag SF and a double flag DF near an outer peripheral edge of the indicia-displaying surface 1637. The distance between each mark is the amount of translation of the second count component 1636 for every revolution of the first count wheel 1640. The indicia-displaying surface 1637 is planar.

The display window 104 in the housing 102 is positioned such that one digit on the first count wheel 1640 and one digit on the second count component 1636 are visible within the display window, and adjacent to each other to form a two-digit number. The displayed digit on the second count component 1636 reflects or tracks the number of ‘tens’ of doses remaining in the inhaler device 100, while the displayed digit on the first count wheel 1640 reflects or tracks the number of ‘ones’ of doses remaining. When the number of remaining doses is below ten, the second count component 1636 will display the single flag SF in the display window 104 instead of or in place of a zero digit. In an embodiment, for example, the single flag SF may be a colored block with no digits thereon to indicate to the user that they are nearing the end of available doses within the inhaler device 100. When the number of remaining doses reaches zero, the second count component 1636 will display the double flag DF in the display window 104 instead of a digit. In an embodiment, for example, the double flag DF may be a colored block with no digits thereon that covers the digit of the first count wheel 1640 to give clear visual feedback to the user that no doses remain within the inhaler device 100. The double flag DF is configured to cover the digit of the first count wheel 1640 when no doses remain within the inhaler device 100.

In the embodiment of FIGS. 16A and 16B, the units or ‘ones’ digits are circumferentially disposed on the first count wheel 1640 in a descending order in the first direction, and the ‘tens’ digits are disposed on the second count component 1636 in a descending order in an upward direction, towards the mouthpiece 110 of the inhaler device. While the counter subassemblies described herein display a number of doses remaining within the inhaler device 100, it will be understood by one of ordinary skill in the art that the counter assemblies may be modified to display a number of doses delivered by the inhaler device by reversing the order of the counting indicia disposed on the counting subassembly.

As best shown on FIG. 16A, the front or indicia-displaying surface 1637 of the second count component 1636 overlaps or overlays a portion of a front or indicia-displaying surface 1641 of the first count wheel 1640. Thus, the ‘ones’ digits on the first count wheel 1640 and the ‘tens’ digits on the second count component 1636 are not coplanar. Stated another way, the ‘ones’ digits on the first count wheel 1640 and the ‘tens’ digits on the second count component 1636 are on different levels, with the ‘tens’ digits being raised relative to the ‘ones’ digits, to be disposed closer to the display window 104 of the inhaler device 100.

Turning now to FIG. 16B, the structure and operation of the first count wheel 1640 and the second count component 1636 will be described in more detail. FIG. 16B is a sectional view taken along line B-B of FIG. 16A, which is along an intermediate position between the indicia-displaying surfaces and the opposing back surfaces of the count subassembly. The relative terms “front” and “back” are utilized herein for sake of illustration only and relate to how an inhaler device is customarily positioned by a user during use with a front of the inhaler device including the display window for the counter mechanism.

The second count component 1636 is a generally rectangular, non-annular component having the front or indicia-displaying surface 1637 and an opposing back surface which includes a plurality of rack teeth 1638, as shown in the sectional view of FIG. 16B. An outer side or surface 1639 extends between the front and back surfaces of the second count component 1636. The outer side 1639 may be stepped, with a smaller width dimension along the back surface of the second count component 1636 and a larger width dimension along the front surface of the second count component. The stepped nature of the outer side 1639 is apparent via a comparison of the relative widths thereof in FIGS. 16A and 16B.

The plurality of rack teeth 1638 are formed on the outer side 1639 of the second count component 136 and do not extend to the front or indicia-displaying surface 1637. In an embodiment, the second count component 1636 includes four rack teeth 1638 but the number of rack teeth is exemplary and depends on the capacity or total number of available doses within the inhaler device 100. The plurality of rack teeth 1638 are longitudinally aligned along a longitudinally-extending axis LA of the inhaler device (shown on FIG. 16B), and each rack tooth 1638 extends outward from the outer side 1639 in a direction towards the single tooth 1642 of the first count wheel 1640.

The second count component 1636 is positioned adjacent or next to the outer circumferential surface 1643 of the first count wheel 1640, such that the single tooth 1642 on the outer edge of the first count wheel 1640 engages or contacts a rack tooth 1638 of the plurality of rack teeth 1638 of the second count component 1636 once per revolution of the first count wheel 1640 to translate the second count component 1636. Stated another way, each time the first count wheel 1640 makes a complete revolution, the single tooth 1642 engages a rack tooth 1638 of the second count component 1636 and moves or translates the second count component 1636 a fixed amount. The first count wheel 1640 indexes the position of the second count component 1636 by driving the second count component 1636 along a linear path. Thus, the second count component 1636 moves a fixed distance linearly once per revolution of the first count wheel 1640. The second count component 1636 is disposed next to or side-by-side in a common plane with the first count wheel 1640. The first count wheel 1640 rotates about a first or central axis of the first count wheel, and the second count component 1636 axially translates along a second axis, i.e., the longitudinally-extending axis LA of the inhaler device (shown on FIG. 16B), the second axis being laterally spaced apart from the first axis. In the embodiment of FIGS. 16A and 16B, the first count wheel 1640 is configured to rotate a counterclockwise direction and the second count component 1636 is configured to move or translate in a downward direction, away from the mouthpiece 110 of the inhaler device. Due to an interaction with the housing 102 which is described in more detail below, the second count component 1636 is stationary and does not translate when the single tooth 1642 of the first count wheel 1640 is not engaged with one of the plurality of rack teeth 1638 of the second count component 1636.

In this embodiment, the housing 102 of the inhaler device 100 includes a flexible arm 1605 that extends from an inner surface thereof. The flexible arm 1605 is configured to interact with the second count component 1636 to maintain the position of the second count component 1636 when the second count component 1636 is not engaged with the first count wheel 1640. The second count component 1636 includes a plurality of notches 1607 on a second outer side or surface 1609 thereof. The second outer surface 1609 opposes the outer surface 1639 of the second count component 1636. The flexible arm 1605 includes a detent 1661 thereon, and the detent 1661 is configured to be received within or mate with each notch 1607 of the plurality of notches 1607 of the second count component 1636. The flexible arm 1605 ensures that the second count component 1636 cannot move away from its current indexed position until it is driven by the first count wheel 1640 with enough force to deflect the flexible arm 1605. The flexible arm 1605 also helps to maintain a consistent position of the second count component 1636 relative to the display window 104 of the inhaler device 100 which results in more consistent alignment between the first and second digits of the two-digit number of the display.

The counter subassembly 1634 may provide a more space efficient layout compared to counters which feature two-wheel components. The counter subassembly 1634 also does not require any type of intermediate components between the count components. By driving the second count component 1636 directly from the first count wheel 1640, the counter subassembly 1634 is simplified and the overall number of component parts of the inhaler device 100 is minimized to reduce the overall cost and complexity of the inhaler device 100. Further, elimination of an intermediate component between the count components means that there is a shorter tolerance chain for alignment between the first count wheel 1640 and the second count component 1636 which results in more consistent alignment between the first and second digits of the two-digit number of the display.

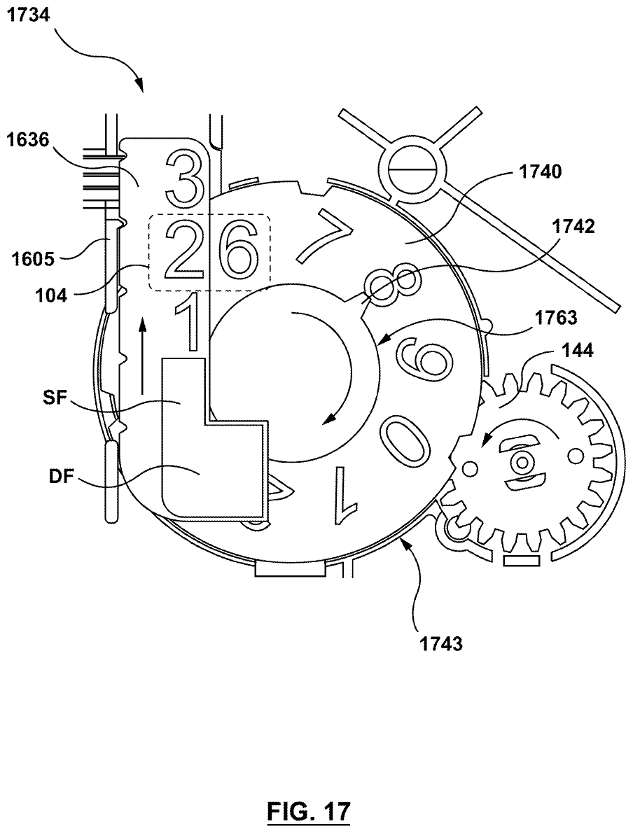

FIG. 17 illustrates another embodiment of a counter subassembly 1734 in which the components thereof are arranged in a different manner than the components of the counter subassembly 1634. The counter subassembly 1734 includes a first count wheel or units ring 1740 and the second count component 1636. The counter subassembly 1734 is the same as the counter subassembly 1634 described above, except for the differences described herein. In this embodiment, the first count wheel 1740 is driven by the dispensing subassembly 120 via gear teeth formed on its outer circumferential side or surface 1743 instead of an inner surface. Stated another way, in the embodiment of FIG. 17, the counter gear (hidden from view in FIG. 17) includes external gear teeth instead of the internal gear teeth of the counter subassembly 134, 1634. The transfer gear 144 meshes with the external gear teeth of the first count wheel 1740, and drives the first count wheel 1740 to rotate in the opposite direction. As described above, when the mouthpiece cover 108 is opened, the transfer gear 144 rotates in the second opposing direction with the second bottom sheet take-up gear 128B. In this embodiment, the transfer gear 144 mates with or directly drives the external gear teeth of the first count wheel 1740 to rotate in the first direction. Thus the first count wheel 1740 rotates in the first direction when the mouthpiece cover 108 is opened.

Since the first count wheel 1740 is configured to rotate in the first direction, the second count component 1636 moves or translates in an upward direction, towards the mouthpiece 110 of the inhaler device. The ‘ones’ digits are circumferentially disposed on the first count wheel 1640 in a descending order in the second opposing direction, and the ‘tens’ digits are disposed on the second count component 1636 in a descending order in a downward direction, away from the mouthpiece 110 of the inhaler device.

In addition, rather than extending from the outer circumferential surface 1743 of the first count wheel 1740, a single tooth 1742 extends from an inner circumferential surface 1763 of the first count wheel 1740. The single tooth 1742 engages or contacts a rack tooth 1638 of the plurality of rack teeth 1638 of the second count component 1636 once per revolution of the first count wheel 1740 to translate the second count component 1636. Stated another way, each time the first count wheel 1740 makes a complete revolution, the single tooth 1742 engages a rack tooth 1638 of the second count component 1636 and moves or translates the second count component 1636 a fixed amount. This arrangement of components with the single tooth 1742 extending from the inner circumferential surface 1763 of the first count wheel 1740 allows the second count component 1636 to overlap or overlay the first count wheel 1740 to a greater extent than the embodiment of FIGS. 16A and 16B, and therefore potentially reduce the overall lateral space taken up by the counter subassembly 1734. There may be restrictions on the space available for the dose counter mechanism, and this embodiment provides a space-efficient arrangement of the components of the counter subassembly. In another embodiment hereof (not shown), the first count wheel 1740 may include more than a single tooth or protrusion 1742. Thus, while at least one single tooth or protrusion is required and a single tooth is deemed to be sufficient and advantageously simplifies the design and reduces material costs of the first count wheel 1740, embodiments hereof may include more than one tooth or protrusion.

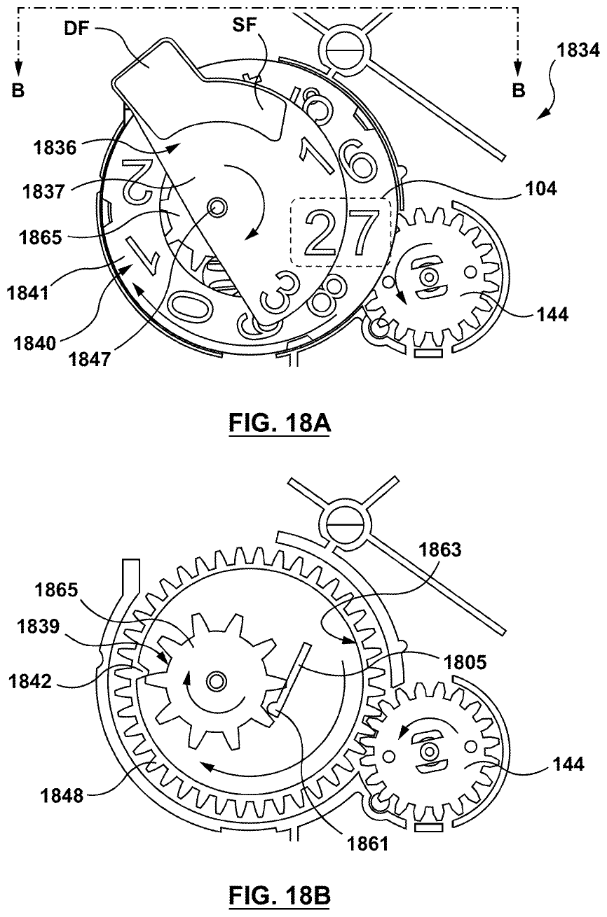

Another embodiment of a counter subassembly 1834 that may be utilized within the inhaler device 100 is depicted in FIGS. 18A and 18B. The counter subassembly 1834 includes a first count wheel or units ring 1840 and a second count component or tens mechanism 1836. Similar to the previous embodiments described herein, the first count wheel 1840, driven from the dispensing subassembly 120 of the inhaler device 100 to rotate a fixed angle per dose, is configured to display a second digit of a two-digit number of the available dose count. The second count component 1836 is driven intermittently from the first count wheel 1840 such that it rotates a fixed angle per revolution of the first count wheel 1840. The second count component 1836 displays a first digit of a two-digit number of the available dose count. As such, the first count wheel 1840 and the second count component 1836 collectively display a number of doses remaining within the inhaler device 100, as shown on FIG. 18A.

A front or indicia-displaying face or surface 1841 of the first count wheel 1840 includes counting indicia disposed thereon that includes units or ‘ones’ digits. More particularly, as shown on FIG. 18A, the counting indicia of the first count wheel 1840 includes the digits 0, 1, 2, 3, 4, 5, 6, 7, 8, 9 in a circular pattern near an outer peripheral edge of the indicia-displaying surface 1841. The angle between each digit is the same as the angle of rotation of the first count wheel 1840 per dose. The indicia-displaying surface 1841 is planar.

A front or indicia-displaying face or surface 1837 of the second count component 1836 includes counting indicia disposed thereon that includes ‘tens’ digits disposed thereon. The counting indicia of the second count component 1836 includes the digits 3, 2, 1 and may also include a single flag SF and a double flag DF near an outer peripheral edge of the indicia-displaying surface 1837. The angle between each mark is the angle of rotation of the second count component 1836 for every revolution of the first count wheel 1840. The indicia-displaying surface 1837 is planar.

Similar to the previous embodiments described herein, the display window 104 in the housing 102 is positioned such that one digit on the first count wheel 1840 and one digit on the second count component 1836 are visible within the display window, and adjacent to each other to form a two-digit number. The displayed digit on the second count component 1836 reflects or tracks the number of ‘tens’ of doses remaining in the inhaler device 100, while the displayed digit on the first count wheel 1840 reflects or tracks the number of ‘ones’ of doses remaining. When the number of remaining doses is below ten, the second count component 1836 will display the single flag SF in the display window 104 instead of or in place of a zero digit. In an embodiment, for example, the single flag SF may be a colored block with no digits thereon to indicate to the user that they are nearing the end of available doses within the inhaler device 100. When the number of remaining doses reaches zero, the second count component 1836 will display the double flag DF in the display window 104 instead of a digit. In an embodiment, for example, the double flag DF may be a colored block with no digits thereon that covers the digit of the first count wheel 1840 to give clear visual feedback to the user that no doses remain within the inhaler device 100. The double flag DF is configured to cover the digit of the first count wheel 1840 when no doses remain within the inhaler device 100.

In the embodiment of FIGS. 18A and 18B, the units or ‘ones’ digits are circumferentially disposed on the first count wheel 1840 in a descending order in the second opposing direction, and the ‘tens’ digits are disposed on the second count component 1836 in a descending order in the second opposing direction. While the counter subassemblies described herein display a number of doses remaining within the inhaler device 100, it will be understood by one of ordinary skill in the art that the counter assemblies may be modified to display a number of doses delivered by the inhaler device by reversing the order of the counting indicia disposed on the counting subassembly.

As best shown on FIG. 18A, the front or indicia-displaying surface 1837 of the second count component 1836 overlaps or overlays a portion of the front or indicia-displaying surface 1841 of the first count wheel 1840. Thus, the ‘ones’ digits on the first count wheel 1840 and the ‘tens’ digits on the second count component 1836 are not coplanar. Stated another way, the ‘ones’ digits on the first count wheel 1840 and the ‘tens’ digits on the second count component 1836 are on different planes or levels, with the ‘tens’ digits being raised relative to the ‘ones’ digits, to be disposed closer to the display window 104.

Turning now to FIG. 18B, the structure and operation of the first count wheel 1840 and the second count component 1836 will be described in more detail. FIG. 18B is a sectional view taken along line B-B of FIG. 18A, which is adjacent to the opposing back surfaces of the count subassembly. The relative terms “front” and “back” are utilized herein for sake of illustration only and relate to how an inhaler device is customarily positioned by a user during use with a front of the inhaler device including the display window for the counter mechanism.

The first count wheel 1840 is an annular or ring-shaped component having the front or indicia-displaying surface 1841 and an opposing back surface which includes a counter gear 1848. Adjacent to the counter gear 1848, a single tooth 1842 extends from an inner circumferential surface 1863 of the first count wheel 1840. The counter gear 1848 may be integrally formed on or attached to the first count wheel 1840. In another embodiment hereof (not shown), the first count wheel 1840 may include more than a single tooth or protrusion 1842. Thus, while at least one single tooth or protrusion is required and a single tooth is deemed to be sufficient and advantageously simplifies the design and reduces material costs of the first count wheel 1840, embodiments hereof may include more than one tooth or protrusion.

The second count component 1836 is configured to rotate around a pivot point 1847. The second count component 1836 is a non-annular or partial-disk component having the front or indicia-displaying surface 1837 and an opposing back surface which includes a second counter gear 1865. The second counter gear 1865 may be integrally formed on or attached to the second count component 1836. The second counter gear 1865 includes a plurality of gear teeth on an outer circumferential surface 1839 thereof, with each gear tooth of the second counter gear 1865 extending radially outward. The second counter gear 1865 of the second count component 1836 is non-concentric with the count gear 1848 of the first count wheel 1840.

The first count wheel 1840 is indirectly driven by the second bottom sheet take-up gear 128B of the dispensing subassembly 120 such that the first count wheel 1840 rotates a fixed angle every time a dose is dispensed. More specifically, in this embodiment, the transfer gear 144 meshes with or directly drives the counter gear 1848 of the first count wheel 1840 and drives the first count wheel 1840 to rotate in the opposite direction. As described above, when the mouthpiece cover 108 is opened, the transfer gear 144 rotates in the second opposing direction with the second bottom sheet take-up gear 128B. As such, in this embodiment, the transfer gear 144 mates with or directly drives the external gear teeth of the first count wheel 1840 to rotate in the first direction. Thus the first count wheel 1840 rotates in the first direction when the mouthpiece cover 108 is opened.

The second count component 1836 is positioned adjacent or next to the inner circumferential surface 1863 of the first count wheel 1840, such that the single tooth 1842 of the first count wheel 1840 engages the second counter gear 1865 of the second count component 1836 once per revolution of the first count wheel 1840 to intermittently rotate the second count component 1836. Stated another way, each time the first count wheel 1840 makes a complete revolution, the single tooth 1842 engages the second counter gear 1865 of the second count component 1836 and turns or rotates the second count component 1836 a fixed amount. Thus, there is a fixed rotation of the second count component 1836 once per revolution of the first count wheel 1840. Since the first count wheel 1840 directly drives the second count component 1836, and the second counter gear 1865 is disposed within the first count wheel 1840, the second count component 1836 rotates in the same direction as the first count wheel 1840. In the embodiment of FIGS. 18A and 18B, each of the first count wheel 1840 and the second count component 1836 is configured to rotate in the first direction. The second count component 1836 is stationary and does not rotate when the single tooth 1842 of the first count wheel 1840 is not engaged with the second counter gear 1865 of the second count component 1836.

The second count component 1836 is disposed within the first count wheel 1840, and in a common plane therewith, but the second count component 1836 rotates on a different axis of rotation than the first count wheel 1840. Stated another way, the first count wheel 1840 rotates about a first axis and the second count component 1836 rotates about a second axis, with the second axis being parallel to and spaced apart from the first axis. There is therefore a fixed rotation of the second count component 1836 once per revolution of the first count wheel 1840, on a different axis of rotation, but in the same direction. In this embodiment, the axis of rotation of the second count component 1836 is within the inner diameter of the first count wheel 1840, which is defined by the inner circumference of the first count wheel 1840. Having the second count component 1836 on a different axis of rotation to the first count wheel 1840 allows the marks on the second count component 1836 to be on a larger radius, which makes it easier to fit larger counting indicia on it to improve readability.

The arrangement of the components of the counter subassembly 1834 potentially reduces the overall lateral space taken up by the counter subassembly 1834 as compared to the counter subassembly 134 described above. There may be restrictions on the space available for the dose counter mechanism, and this embodiment provides a space-efficient arrangement of the components of the counter subassembly. The counter subassembly 1834 also does not require any type of intermediate components between the count components. By driving the second count component 1836 directly from the first count wheel 1840, the counter subassembly 1834 is simplified and overall component count of the inhaler device 100 is minimized to reduce the overall cost and complexity of the inhaler device 100. Further, elimination of an intermediate component between the count components means that there is a shorter tolerance chain for alignment between the first count wheel 1840 and the second count component 1836 which results in more consistent alignment between the first and second digits of the two-digit number of the display.

In this embodiment, a back plate of the counter subassembly 1845 includes a flexible arm 1805. The flexible arm 1805 is configured to interact with the second count component 1836 to maintain the position of the second count component 1836 when the second count component 1836 is not engaged with the first count wheel 1840. The flexible arm 1805 includes a detent 1861 thereon, and the detent 1861 is configured to be received within or mate with a notch formed between adjacent gear teeth of the second counter gear 1865. The flexible arm 1805 ensures that the second count component 1836 cannot move away from its current indexed position until it is driven by the first count wheel 1840 with enough force to deflect the flexible arm 1805. The flexible arm 1805 also helps to maintain a consistent position of the second count component 1836 relative to the display window 104 of the inhaler device 100 which results in more consistent alignment between the first and second digits of the two-digit number of the display.

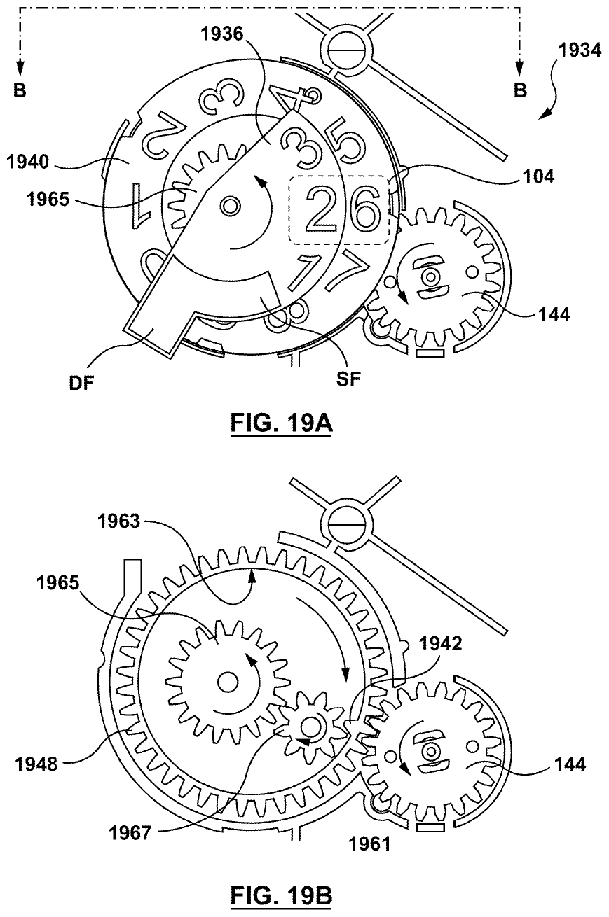

Although there are advantages to the elimination of an intermediate component between the components of the count subassembly as described above, use of an intermediate component may potentially make the design of a counter subassembly more flexible for different gear ratios and component positions. Since the design of a dose counter mechanism may be dependent on other elements of the inhaler, such as the design of the housing and the layout of the dispensing subassembly 120, increased flexibility of the design of a counter subassembly is desirable to find an arrangement that best suits other design elements. Accordingly, FIGS. 19A and 19B illustrates another embodiment of a counter subassembly 1934 that includes an intermediate component. The counter subassembly 1934 includes a first count wheel or units ring 1940 and a second count component 1936. The counter subassembly 1934 is the same as the counter subassembly 1834 described above, except for the differences described herein. In this embodiment, an intermediate counter gear 1967 is disposed between a first counter gear 1948 formed with the first count wheel 1940 and a second counter gear 1965 formed with the second count component 1936. The intermediate counter gear 1967 transfers rotation of the first count wheel 1940 to the second count component 1936, instead of the two components directly meshing with each other.

Similar to the first count wheel 1840 described above, the first count wheel 1940 is driven by the second bottom sheet take-up gear 128B of the dispensing subassembly 120 such that the first count wheel 1940 rotates a fixed angle every time a dose is dispensed. As described above, when the mouthpiece cover 108 is opened, the transfer gear 144 rotates in the second opposing direction with the second bottom sheet take-up gear 128B. As such, in this embodiment, the transfer gear 144 mates with or directly drives the external gear teeth of an external gear 1948 of the first count wheel 1940 to rotate in the first direction. Thus the first count wheel 1940 rotates in the first direction when the mouthpiece cover 108 is opened.

The intermediate counter gear 1967 is positioned adjacent or next to an inner circumferential surface 1963 of the first count wheel 1940, such that a single tooth 1942 of the first count wheel 1940 engages the intermediate counter gear 1967 once per revolution of the first count wheel 1940. The intermediate counter gear 1967 is also positioned adjacent or next to the second counter gear 1965 of the second count component 1936 such that the second counter gear 1965 rotates with the intermediate counter gear 1967. The second counter gear 1965 of the second count component 1936 is always engaged with the intermediate counter gear 1967, so the second count component 1936 rotates whenever the intermediate counter gear 1967 rotates. Thus, each time that the first count wheel 1940 makes a complete revolution, the single tooth 1942 engages the intermediate counter gear 1967 and the second count component 1936 is rotated or turned a fixed amount. Thus, there is a fixed rotation of the second count component 1936 once per revolution of the first count wheel 1940. The first count wheel 1940 thus indirectly drives the second count component 1936 in this embodiment, via the intermediate counter gear 1967. In the embodiment of FIGS. 19A and 19B, each of the first count wheel 1940 and the intermediate counter gear 1967 is configured to rotate in the first direction and the second count component 1936 is configured to rotate in the second opposing direction. The second count component 1936 is stationary and does not rotate when the single tooth 1942 of the first count wheel 1940 is not engaged with intermediate counter gear 1967. In another embodiment hereof (not shown), the first count wheel 1940 may include more than a single tooth or protrusion 1942. Thus, while at least one single tooth or protrusion is required and a single tooth is deemed to be sufficient and advantageously simplifies the design and reduces material costs of the first count wheel 1940, embodiments hereof may include more than one tooth or protrusion.

In the embodiment of FIGS. 19A and 19B, the second count component 1936 rotates in an opposite direction than the first count wheel 1940 and the intermediate counter gear 1967. As such, as shown in FIG. 19A, the units or ‘ones’ digits are circumferentially disposed on the first count wheel 1940 in a descending order in the second opposing or counterclockwise direction, and the ‘tens’ digits are disposed on the second count component 1836 in a descending order in the first or clockwise direction.

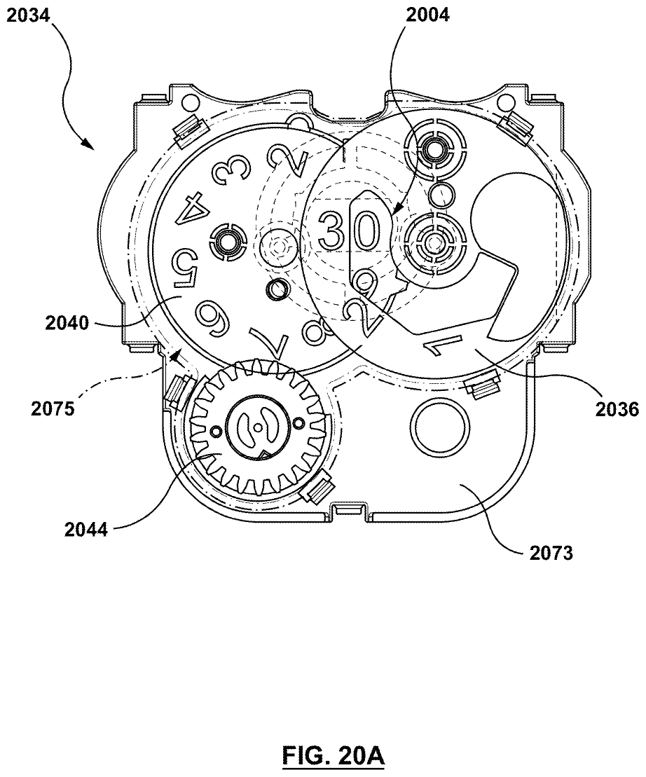

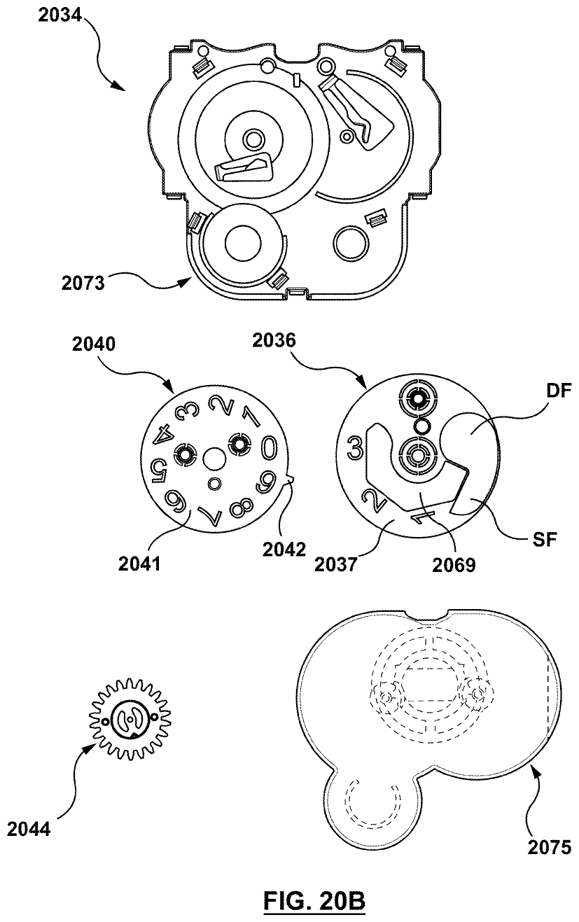

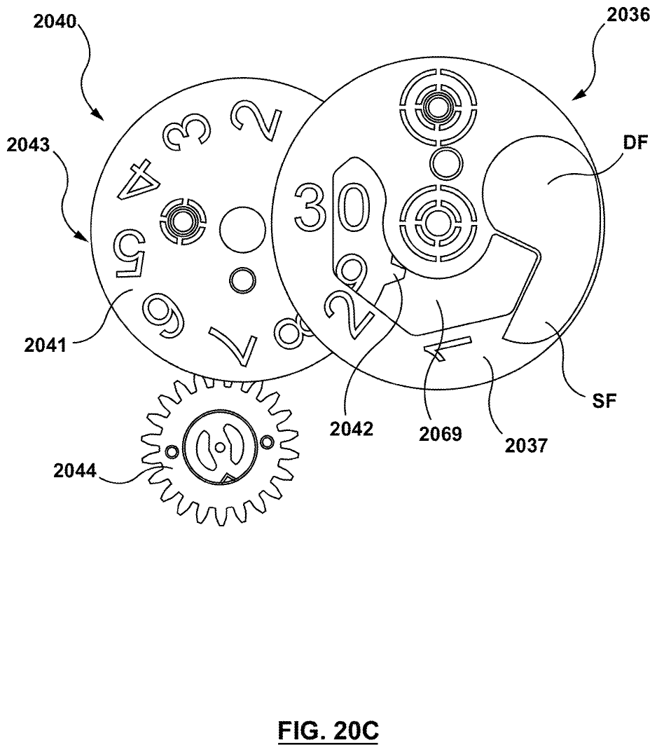

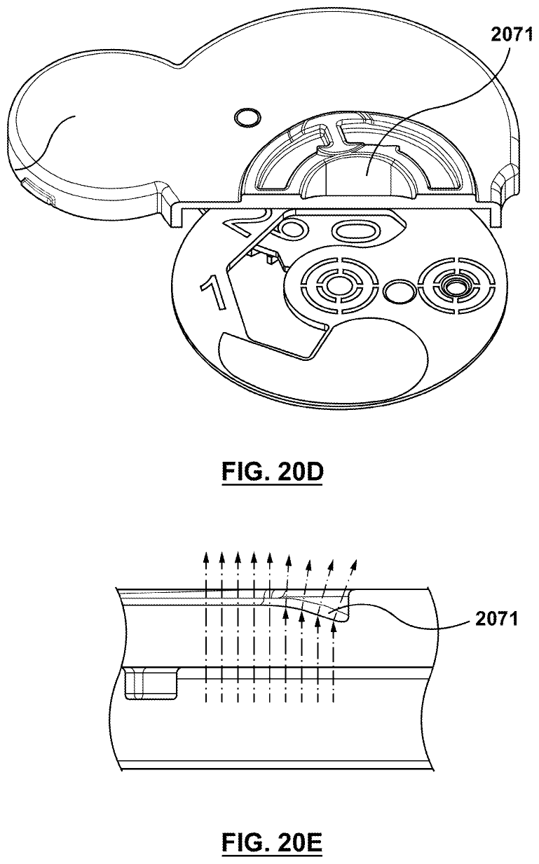

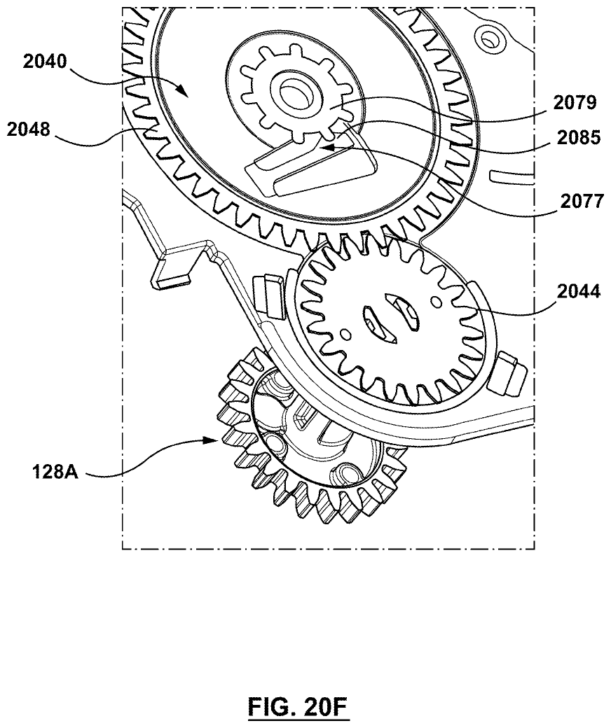

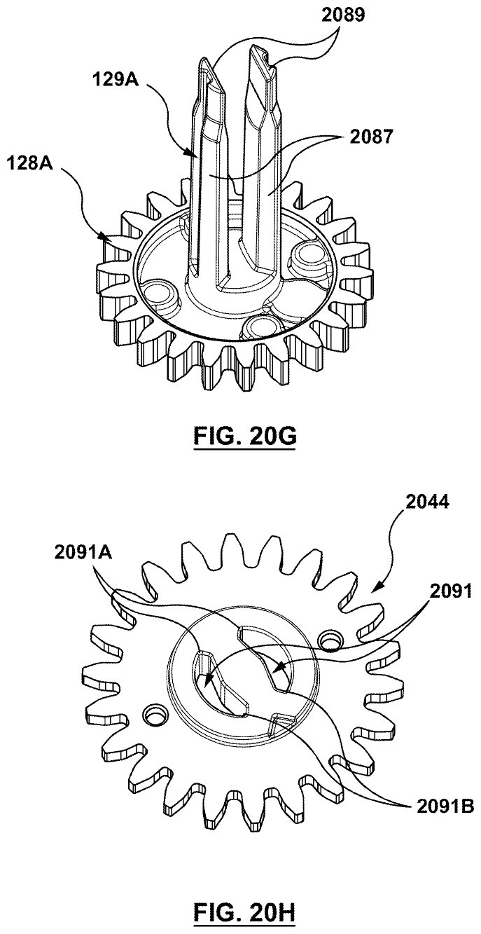

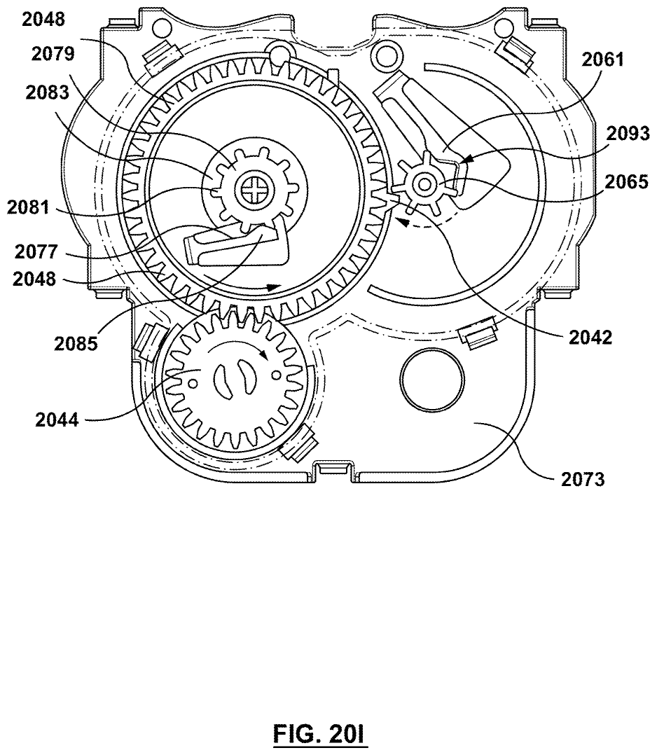

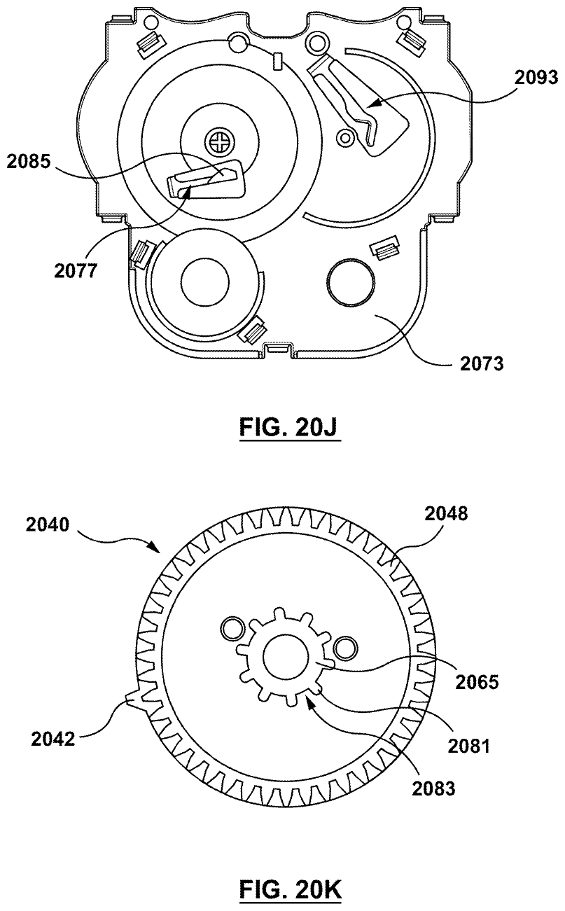

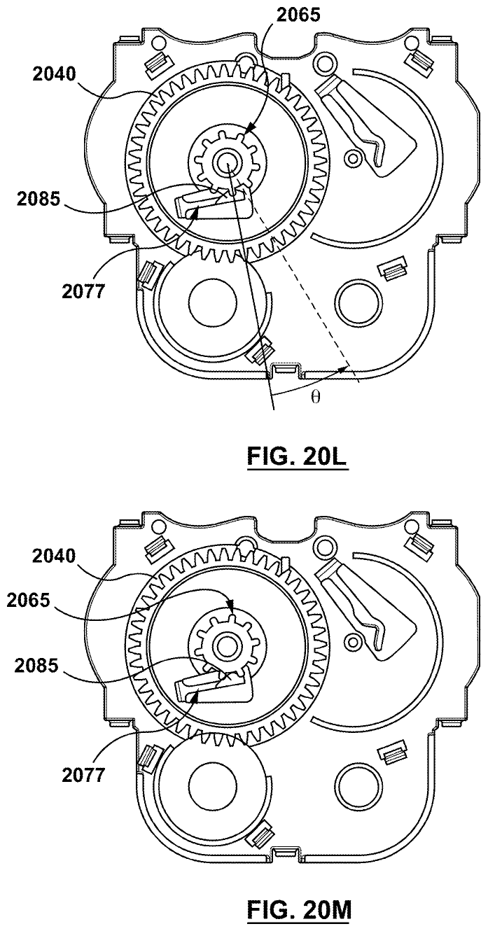

Another embodiment of a counter subassembly 2034 that may be utilized within the inhaler device 100 is depicted in FIGS. 20A-20M. FIG. 20A illustrates the counter subassembly 2034 as assembled, while FIG. 20B illustrates an exploded view of the components of the counter subassembly 2034. The counter subassembly 2034 includes a first count wheel or units ring 2040 and a second count wheel or tens ring 2036. Similar to the previous embodiments described herein, the first count wheel 2040, driven from the dispensing subassembly 120 of the inhaler device 100 to rotate a fixed angle per dose, is configured to display a second digit of a two-digit number of the available dose count. The second count wheel 2036 is driven intermittently from the first count wheel 2040 such that it rotates a fixed angle per revolution of the first count wheel 2040. The second count wheel 2036 displays a first digit of a two-digit number of the available dose count. As such, the first count wheel 2040 and the second count wheel 2036 collectively display a number of doses remaining within the inhaler device 100, as shown on FIG. 20A. In addition to the first count wheel 2040 and the second count wheel 2036, the counter subassembly 2034 includes a back plate 2073, a cover 2075, and a transfer gear 2044. When assembled as shown in FIG. 20A, the first count wheel 2040, the second count wheel 2036, and the transfer gear 2044 are disposed between the back plate 2073 and the cover 2075, such that the cover 2075 overlaps or overlays the first count wheel 2040, the second count wheel 2036, and the transfer gear 2044.

A front or indicia-displaying face or surface 2041 of the first count wheel 2040 includes counting indicia disposed thereon that includes units or ‘ones’ digits. More particularly, as best shown on the exploded view of FIG. 20B, the counting indicia of the first count wheel 2040 includes the digits 0, 1, 2, 3, 4, 5, 6, 7, 8, 9 in a circular pattern near an outer peripheral edge of the indicia-displaying surface 2041. The angle between each digit is the same as the angle of rotation of the first count wheel 2040 per dose. The indicia-displaying surface 2041 is planar.

A front or indicia-displaying face or surface 2037 of the second count wheel 2036 includes counting indicia disposed thereon that includes ‘tens’ digits disposed thereon. The counting indicia of the second count wheel 2036 includes the digits 3, 2, 1 and may also include a single flag SF and a double flag DF near an outer peripheral edge of the indicia-displaying surface 2037. The angle between each mark is the angle of rotation of the second count wheel 2036 for every revolution of the first count wheel 2040. The indicia-displaying surface 2037 is planar.

Similar to the previous embodiments described herein, a display window 2004 in the housing 102 is positioned such that one digit on the first count wheel 2040 and one digit on the second count wheel 2036 are visible within the display window, and adjacent to each other to form a two-digit number. In this embodiment, the display window 2004 is generally circular, as will be described in more detail herein. The displayed digit on the second count wheel 2036 reflects or tracks the number of ‘tens’ of doses remaining in the inhaler device 100, while the displayed digit on the first count wheel 2040 reflects or tracks the number of ‘ones’ of doses remaining. When the number of remaining doses is below ten, the second count wheel 2036 will display the single flag SF in the display window 2004 instead of or in place of a zero digit. In an embodiment, for example, the single flag SF may be a colored block with no digits thereon to indicate to the user that they are nearing the end of available doses within the inhaler device 100. When the number of remaining doses reaches zero, the second count wheel 2036 will display the double flag DF in the display window 2004 instead of a digit. In an embodiment, for example, the double flag DF may be a colored block with no digits thereon that covers the digit of the first count wheel 2040 to give clear visual feedback to the user that no doses remain within the inhaler device 100. The double flag DF is configured to cover the digit of the first count wheel 2040 when no doses remain within the inhaler device 100.

In the embodiment of FIGS. 20A-20M, the units or ‘ones’ digits are circumferentially disposed on the first count wheel 2040 in a descending order in the first direction, and the ‘tens’ digits are disposed on the second count wheel 2036 in a descending order in the second opposing direction. While the counter subassemblies described herein display a number of doses remaining within the inhaler device 100, it will be understood by one of ordinary skill in the art that the counter assemblies may be modified to display a number of doses delivered by the inhaler device by reversing the order of the counting indicia disposed on the counting subassembly.

As best shown on FIG. 20C, the front or indicia-displaying surface 2047 of the second count wheel 2036 overlaps or overlays a portion of the front or indicia-displaying surface 2041 of the first count wheel 2040. Thus, the ‘ones’ digits on the first count wheel 2040 and the ‘tens’ digits on the second count wheel 2036 are not coplanar. Stated another way, the ‘ones’ digits on the first count wheel 2040 and the ‘tens’ digits on the second count wheel 2036 are on different planes or levels, with the ‘tens’ digits being raised relative to the ‘ones’ digits, to be disposed closer to the display window 2004.

The structure and operation of the first count wheel 2040 and the second count wheel 2036 will be described in more detail. The first count wheel 2040 is an annular or ring-shaped component having the front or indicia-displaying surface 2041 and an opposing back surface which includes a first counter gear 2048, as shown on FIGS. 20F, 20I, 20K, 20L, and 20M. The first counter gear 2048 may be integrally formed on or attached to the first count wheel 140. An outer circumferential surface or side 2043 extends between the front and back surfaces of the first count wheel 140. The outer circumferential side 2043 may be stepped, with a smaller diameter along the back surface of the first count wheel 2040 and a larger diameter along the front surface of the first count wheel 2040. The indicia-displaying surface 2041 of the first count wheel 2040 thus has a greater outer diameter than the first counter gear 2048.

The front or indicia-displaying surface 2041 of the first count wheel 2040 includes a single tooth or protrusion 2042 extending radially outward from the outer circumferential side 2043. In another embodiment hereof (not shown), the first count wheel 2040 may include more than a single tooth or protrusion 2042. Thus, while at least one single tooth or protrusion is required and a single tooth is deemed to be sufficient and advantageously simplifies the design and reduces material costs of the first count wheel 2040, embodiments hereof may include more than one tooth or protrusion.

The second count wheel 2036 is an annular or ring-shaped component having the front or indicia-displaying surface 2037 and an opposing back surface which includes a second counter gear 2065, which is shown on FIG. 20I. The second counter gear 2065 may be integrally formed on or attached to the second count wheel 2036. The second counter gear 2065 includes a plurality of gear teeth on an outer circumferential surface thereof, with each gear tooth of the second counter gear 2065 extending radially outward. The second counter gear 2065 of the second count wheel 2036 is non-concentric with the count gear 2048 of the first count wheel 2040.

As best shown on FIG. 20C, in this embodiment, the second count wheel 2036 overlaps or overlays a portion of the first count wheel 2040 and the second count wheel 2036 contain a cut-out 2069 such that the “ones” digit of the first count wheel 2040 is visible through the cut-out 2069 of the second count wheel 2036. The geometry of the cut-out 2069 is exemplary and includes one continuous slot disposed radially inwards of the counting indicia of the second count wheel 2036, but may alternatively include a plurality or series of holes such that a hole is disposed radially inwards of each counting indicia of the second count wheel 2036. The single tooth 2042 of the first count wheel 2040 engages the second counter gear 2065 of the second count wheel 2036 once per revolution of the first count wheel 2040 to intermittently rotate the second count wheel 2036. Stated another way, each time the first count wheel 2040 makes a complete revolution, the single tooth 2042 engages the second counter gear 2065 of the second count wheel 2036 and turns or rotates the second count wheel 2036 a fixed amount. Thus, there is a fixed rotation of the second count wheel 2036 once per revolution of the first count wheel 2040. The second count wheel 2036 rotates on a different axis of rotation than the first count wheel 2040. Stated another way, the first count wheel 2040 rotates about a first axis and the second count wheel 2036 rotates about a second axis, with the second axis being parallel to and spaced apart from the first axis. Having the second count wheel 2036 on a different axis of rotation relative to the first count wheel 2040 allows the counting indicia on the second count wheel 2036 to be on a larger radius, which makes it easier to fit larger counting indicia on it to improve readability.

As best shown on FIG. 20C, the spacing between the counting indicia on the second count wheel 2036 is greater than the spacing of the counting indicia on the first count wheel 2040. The increased spacing of the counting indicia on the second count wheel 2036 lowers the risk of the user seeing adjacent indicia of the second count wheel 2036 through the display window 2004. Further, since the second count wheel 2036 overlaps or overlays a portion of the first count wheel 2040, the second count wheel 2036 covers adjacent indicia on the first count wheel 2040 and lowers the risk of the user seeing adjacent indicia of the first count wheel 2040 through the display window 2004. Reducing visibility of adjacent counting indicia on the count wheels permits greater flexibility in the shape of the display window 104. For example, as shown in the embodiment of FIGS. 20A-20M, the display window 2004 of the inhaler device is circular rather than rectangular, which may improve visibility of the counting indicia when the inhaler device is viewed from above or below the display window, i.e., at an angle which is not perpendicular to the front of the inhaler device.

The arrangement of the components of the counter subassembly 2034 potentially reduces the overall lateral space taken up by the counter subassembly 2034 as compared to the counter subassembly 134 described above. There may be restrictions on the space available for the dose counter mechanism, and this embodiment provides a space-efficient arrangement of the components of the counter subassembly. Particularly, since the second count wheel 2036 overlaps or overlays a portion of the first count wheel 2040, the axes of rotation of the count wheels are closer together as compared to the counter subassembly 134 described above, thus reducing the overall width of the counter subassembly and/or maximizing the size of the count wheels (and therefore count indicia) within the available width or space for the counter subassembly.

The counter subassembly 2034 also does not require any type of intermediate components between the count wheels. By driving the second count wheel 2036 directly from the first count wheel 2040, the counter subassembly 2034 is simplified and overall component count of the inhaler device 100 is minimized to reduce the overall cost and complexity of the inhaler device 100. Further, elimination of an intermediate component between the count wheels means that there is a shorter tolerance chain for alignment between the first count wheel 2040 and the second count wheel 2036 which results in more consistent alignment between the first and second digits of the two-digit number of the display.

With reference to FIGS. 20D and 20E, a lens 2071 may be positioned in front of the counting indicia in the region of the display window 2004. The lens 2071 may improve visibility of the counting indicia when the inhaler device is viewed from above or below the display window, i.e., at an angle which is not perpendicular to the front of the inhaler device. The depth of the lens 2071 is maximized to reduce the perceived distance between the front of the inhaler housing and the front of the count wheels. Further, as shown in FIG. 20E, the lens 2071 may be non-planar to reduce visibility in peripheral areas of the window. In an embodiment, a peripheral edge of the lens 2071 may be frosted to reduce visibility of areas of the display window 2004 away from the target counting indicia. In an embodiment, the lens 2071 may also integrate magnification to maximize the perceived height of the counting indicia.

With reference to FIGS. 20F-20H, in this embodiment, the first count wheel 2040 is driven by the first bottom sheet take-up gear 128A of the dispensing subassembly 120 rather than the second bottom sheet take-up gear 128B. More specifically, in this embodiment as best shown in FIG. 20F, the transfer gear 2044 is attached to an opposing end of the first spindle 129A of the first bottom sheet take-up gear 128A so that the transfer gear 2044, the first spindle 129A, and the first bottom sheet take-up gear 128A rotate simultaneously as an assembly. When the mouthpiece cover 108 is opened, the transfer gear 2044 thus rotates in the first direction with the first bottom sheet take-up gear 128A. The transfer gear 2044 mates with or directly drives the first counter gear 2048 to rotate in the second opposing direction. Thus, in this embodiment, the first counter gear 2048 rotates in the opposite direction as the transfer gear 2044 and the first count wheel 2040 rotates in the second opposing direction when the mouthpiece cover 108 is opened. Similar to the previous embodiments, the first count wheel 2040 rotates a fixed angle every time a dose is dispensed.

As best shown on FIG. 20I, the first count wheel 2040 directly drives the second count wheel 2036 via interaction between the single tooth 2042 and the second counter gear 2065 such that the second count wheel 2036 rotates in the opposite direction from the first count wheel 2040. Thus, the first count wheel 2040 rotates in the second opposing direction (i.e., counter clockwise) and the second count wheel 2036 rotates in the first direction (i.e., clockwise). The second count wheel 2036 is stationary and does not rotate when the single tooth 2042 of the first count wheel 2040 is not engaged with the second counter gear 2065 of the second count wheel 2036. More particularly, in this embodiment, a second flexible arm 2093 is integrally formed on or attached to the back plate 2073 and is configured to interact with the second counter gear 2065 disposed on the back surface of the second count wheel 2036. The second flexible arm 2093 is configured to interact with the second count component 2036 to maintain the position of the second count component 2036 when the second count component 2036 is not engaged with the first count wheel 2040. Stated another way, the second flexible arm 2093 holds or retains the second count wheel 2036 in alignment when not being driven by the first count wheel 2040. As such, the second flexible arm 2093 reduces variation in position of the counting indicia on the second count wheel 2036 which may result from component tolerances. The second flexible arm 2093 includes a detent 2061 thereon, and the detent 2061 is configured to extend between adjacent gear teeth of the second counter gear 2065 of the second count component 2036. The second flexible arm 2093 ensures that the second count component 2036 cannot move away from its current indexed position until it is driven by the first count wheel 2040 with enough force to deflect the second flexible arm 2093. The second flexible arm 2093 also helps to maintain a consistent position of the second count component 2036 relative to the display window 2004 of the inhaler device which results in more consistent alignment between the first and second digits of the two-digit number of the display.

In this embodiment, a detent feature is also added to counter subassembly 2034 in order to ensure rotational accuracy with which the counter subassembly 2034 is driven as the mouthpiece cover 108 is opened. The detent feature is configured to drive the first count wheel 2040 to its final position once it has been driven to within a certain rotation of the target nominal orientation of the first count wheel 2040 by the dispensing subassembly 120, but the detent feature does not have enough force to rotate the entire gear train (i.e., the gear train of the dispensing subassembly 120) leading up to the first count wheel 2040 therewith. The detent feature accommodates variations in the tolerance of the gear train of the dispensing subassembly 120.

More particularly, with reference to FIGS. 20I-20M, the detent feature includes a first flexible arm 2077 that is integrally formed on or attached to the back plate 2073 and is configured to interact with a detent wheel 2079 disposed on the back surface of the first count wheel 2040. The detent wheel 2079 may be concentric with the first counter gear 2048. The detent wheel 2079 includes a plurality of radially-extending rounded protrusions 2081, with a notch or gap 2083 being formed between each pair of adjacent rounded protrusions 2081. The first flexible arm 2077 includes a detent 2085 that is configured to be received within a notch 2083 of the detent wheel 2079.

The first flexible arm 2077 is configured to pull or rotate the first count wheel 2040 and thereby control the position of the first count wheel 2040. With particular reference to FIG. 20L, rotation of the first count wheel 2040 is driven by the mouthpiece cover 108 and the dispensing subassembly 120 up to a target nominal position, θ°, and thereafter rotation of the first count wheel 2040 is driven by the first flexible arm 2077. More particularly, as the mouthpiece cover 108 is opened to and as far as when the first count wheel 2040 reaches θ° before the final position, the transfer gear 2044 rotates in the first direction (i.e., clockwise) with the first bottom sheet take-up gear 128A and thereby causes the first count wheel 2040 to rotate in the second opposing direction (i.e., counter clockwise) as described above. After the first count wheel 2040 reaches the target nominal position or θ°, the first flexible arm 2077 causes the first count wheel 2040 to further rotate in the second opposing direction (i.e., counter clockwise) because the detent 2085 of the first flexible arm 2077 is biased to be received within a notch 2083 of the detent wheel 2079. When the detent 2085 of the first flexible arm 2077 moves into a notch 2083 of the detent wheel 2079 as shown in FIG. 20M, the first flexible arm 2077 pulls the first count wheel 2040 in the second direction (i.e., counter clockwise) to the final position and thereby drives the transfer gear 2044 in the first direction (i.e., clockwise) ahead of, or prior to, rotation of the first bottom sheet take-up gear 128A. Thus, the first flexible arm 2077 drives and secures the first count wheel 2040 accurately in target position, and is independent from the mouthpiece cover 108 and the dispensing subassembly 120 tolerance chain leading up to it.

In order for the detent feature to operate properly, the inhaler device must include a suitably large clearance or backlash that allows the counter subassembly 2034 to rotate forwards, i.e., in the second opposing direction, without the gear train of the dispensing subassembly 120. In the embodiment of FIGS. 20A-20M, a suitable backlash or clearance is provided at the interface between the first bottom sheet take-up gear 128A and the transfer gear 2044. As described above with respect to FIGS. 20F-20H, the transfer gear 2044 is attached to an opposing end of the first spindle 129A of the first bottom sheet take-up gear 128A. More particularly, the first spindle 129A includes a pair of upstands or posts 2087 with free or unattached ends 2089. The free or unattached ends 2089 of the posts 2087 interface with relatively larger apertures 2091 of the transfer gear 2044. The loose fit between the posts 2087 and the apertures 2091 provides the suitable backlash or clearance for the detent feature to operate properly. More particularly, when the transfer gear 2044 rotates in the first direction (i.e., clockwise) with the first bottom sheet take-up gear 128A, the posts 2087 of the first bottom sheet take-up gear 128A contact the transfer gear 2044 at the driving faces 2091A of the apertures 2091. Conversely, when the first flexible arm 2077 pulls the first count wheel 2040 in the second direction (i.e., counter clockwise) to the final position and thereby drives the transfer gear 2044 in the first direction (i.e., clockwise) ahead of, or prior to, rotation of the first bottom sheet take-up gear 128A, there is sufficient clearance within the apertures 2091 of the transfer gear 2044 to permit the free or unattached ends 2089 of the posts 2087 to move towards the non-driving faces 2091B of the apertures 2091.

Although detent feature including the first flexible arm 2077 is depicted only in the embodiment of FIGS. 20A-20M, it will be apparent to those of ordinary skill in the art that this detent feature may be incorporated into counter subassemblies 134, 1534, 1634, 1734, and/or 1834 to ensure rotational accuracy of the respective first count wheel.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

1. A dry powder inhaler device comprising:
an actuator for operating a dispensing mechanism of the dry powder inhaler device;
a first count wheel configured to be rotated by the dispensing mechanism, wherein the first count wheel includes a single tooth extending radially outward from an outer circumferential surface or side thereof; and
a second count component disposed adjacent to the outer circumferential surface or side of the first count wheel, wherein the second count component includes a plurality of notches on an outer surface or side thereof, each notch extending radially inward and each notch configured to mate with the single tooth,
wherein the single tooth of the first count wheel engages one of the plurality of notches of the second count component once per revolution of the first count wheel to intermittently rotate the second count component, the first count wheel and the second count component being configured to rotate in opposing directions, and
wherein the first count wheel and the second count component collectively display a number of doses remaining within the inhaler device or a number of doses delivered by the inhaler device.

2. The dry powder inhaler device of claim 1, wherein the actuator is a mouthpiece cover.

3. The dry powder inhaler device of claim 1, wherein the first count wheel rotates about a first axis and the second count component rotates about a second axis, the second axis being parallel to and spaced apart from the first axis.

4. The dry powder inhaler device of claim 1, wherein the plurality of notches includes four notches.

5. The dry powder inhaler device of claim 1, wherein a segment of the outer surface or side of the second count component extending between two adjacent notches of the plurality of notches is concave and forms an arc-shaped indent that is coincident with the outer circumferential surface or side of the first count wheel.

6. The dry powder inhaler device of claim 1, wherein the outer surface or side of the second count component includes a plurality of segments, each segment extending between two adjacent notches of the plurality of notches, and wherein each segment is concave and forms an arc-shaped indent that is coincident with the outer circumferential surface or side of the first count wheel.

7. The dry powder inhaler device of claim 1, wherein the first count wheel is an annular component.

8. The dry powder inhaler device of claim 1, wherein the second count component is a non-annular component.

9. The dry powder inhaler device of claim 1, wherein an indicia-displaying surface of the first count wheel includes ‘ones’ digits disposed thereon and an indicia displaying surface of the second count component includes ‘tens’ digits disposed thereon.

10. The dry powder inhaler device of claim 9, wherein the indicia-displaying surfaces of the first count wheel and the second count component are coplanar.

11. The dry powder inhaler device of claim 9, wherein the indicia-displaying surfaces of the first count wheel and the second count component are not coplanar, with the indicia displaying surface of the second count component disposed closer to a display window of the inhaler device than the indicia displaying surface of the first count wheel.

12. The dry powder inhaler device of claim 1, wherein the second count component is configured to rotate in a first direction and the first count wheel is configured to rotate in a second opposing direction.

13. The dry powder inhaler device of claim 1, wherein the second count component includes a single flag in place of a zero, the single flag being a colored block with no digits thereon.

14. The dry powder inhaler device of claim 1, wherein the second count component includes a double flag, the double flag being a colored block with no digits thereon, and wherein the double flag is configured to cover a digit of the first count wheel when no doses remain within the inhaler device.

15. The dry powder inhaler device of claim 1, wherein the outer circumferential surface or side of the first count wheel is generally circular with a clearance gap around the single tooth.

16. The dry powder inhaler device of claim 1, wherein the second count component is stationary when the single tooth of the first count wheel is not engaged within one of the plurality of notches of the second count component.

17. The dry powder inhaler device of claim 1, wherein the first count wheel includes detent wheel having a plurality of notches and wherein the dry powder inhaler device further includes a housing and a flexible arm extending from a housing, the flexible arm including a detent thereon, the detent being configured to be received within a notch of the plurality of notches of the first count wheel.

18. The dry powder inhaler device of claim 17, wherein the flexible arm is configured to drive and secure the first count wheel within a target position.

19. The dry powder inhaler device of claim 1, wherein the dry powder inhaler device further includes a housing and a flexible arm extending from a housing, the flexible arm including a detent thereon, the detent being configured to be received within a notch of the plurality of notches of the second count component.

20. A dry powder inhaler device comprising:
an actuator for operating a dispensing mechanism of the dry powder inhaler device;
a first count wheel configured to be rotated by the dispensing mechanism, wherein the first count wheel includes a single tooth extending radially outward from a circumferential surface or side thereof; and
a second count component disposed adjacent to the first count wheel, wherein the second count component includes a plurality of rack teeth on an outer surface or side thereof, the plurality of rack teeth longitudinally aligned and extending outwards in a direction towards the single tooth of the first count wheel,
wherein the single tooth of the first count wheel engages one of the plurality of rack teeth of the second count component once per complete revolution of the first count wheel to intermittently translate the second count component, and
wherein the first count wheel and the second count component collectively display a number of doses remaining within the inhaler device or a number of doses delivered by the inhaler device.

21-47. (canceled)
